(19) United States
(12) Patent Application Publication
(10) Pub. No.: US 2011/0027328 A1
(43) Pub. Date: Feb. 3, 2011

(54) Oral Care Articles and Methods

(76) Inventors: Arif Ali Baig, Mason, OH (US); Robert Wayne Glen, JR., Liberty Township, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/846,862

(22) Filed: Jul. 30, 2010

Related U.S. Application Data
(60) Provisional application No. 61/229,794, filed on Jul. 30, 2009.

(51) Int. Cl. A61K 8/02; A61Q 11/00

(52) U.S. Cl. 424/401; 424/49

(57) ABSTRACT

An oral care article in the form of a flexible porous dissolvable solid structure, comprising: from about 1% to about 70% surfactant; from about 10% to about 70% water soluble polymer, from about 0% to about 25% plasticizer; and wherein said article comprises an oral care component and has a density of from about 0.03 g/cm³ to about 0.5 g/cm³.

### CROSS REFERENCE TO RELATED APPLICATION

This application claims the benefit of U.S. Provisional Application No. 61/229,794, filed Jul. 30, 2009, the entire substance of which is incorporated herein by reference.

### FIELD OF THE INVENTION

Discussed herein are oral care articles, oral care compositions, devices containing articles, and methods relating thereto.

### BACKGROUND OF THE INVENTION

Many oral care products in the market today are sold containing water. The water in the formula adds to the weight and size of the products and translates into greater shipping and storage costs. Additionally, these types of products also have disadvantages in terms of packaging, storage, transportation, and convenience of use. For example, liquid oral care products are typically packaged in bottles, paste products are typically packaged in tubes, and gel products come in many forms like tubes or, for example, on a carrier material such as a strip. This packaging can contribute to added cost of the product as well as added waste which often ends up in landfills. It can also be difficult to control the dosing of liquid and paste products. Moreover, the presence of water in oral care compositions increases susceptibility to degradation of water unstable ingredients and promotes negative interactions between two or more incompatible materials in a single phase composition. Therefore, a need exists for oral care products which can alleviate some of the inconveniences and costs of traditional water containing oral care products.

### SUMMARY OF THE INVENTION

The present invention relates to an oral care article in the form of a flexible porous dissolvable solid structure, comprising: from about 1% to about 70% surfactant; from about 10% to about 70% water soluble polymer, from about 0% to about 25% plasticizer; and wherein said article comprises an oral care component and has a density of from about 0.03 g/cm³ to about 0.5 g/cm³.

The present invention further relates to a process for making an oral care article in the form of a porous dissolvable solid structure. The article is formed by a process comprising the steps of: preparing a pre-mix comprising surfactant, dissolved polymer structurant, and optionally plasticizer, wherein said pre-mix has from about 10% to about 70% total solids and a viscosity of from about 2,500 cps to 150,000 cps; aerating said pre-mix by introducing a gas into the pre-mix to form a wet aerated pre-mix; forming the wet aerated pre-mix into a desired one or more shapes to form shaped wet pre-mix; and drying the shaped wet pre-mix to a desired final moisture content from about 0.5% to about 15% moisture, to form the oral care article.

### BRIEF DESCRIPTION OF THE DRAWINGS

The accompanying figures show non-limiting embodiments of the present invention.

FIG. 1 is a schematic view of a porous dissolvable solid structure with a surface resident coating.

FIG. 2 is a schematic view of two porous dissolvable solid structures with a surface resident coating in between the two structures.

FIGS. 3A and 3B are schematic views of a dimpled porous dissolvable solid structure with a surface resident coating inside the dimples.

FIG. 4 is a schematic view of a porous dissolvable solid structure that is folded over to enclose a surface resident coating.

### DETAILED DESCRIPTION OF THE INVENTION

### Definitions

The present inventors have found that dissolvable solid oral care articles can be prepared that can be conveniently and quickly dissolved to reconstitute a liquid product for ease of application to the oral cavity while providing sufficient topical delivery of active agents to the oral cavity (with similar performance as conventional liquid oral care products).

By “oral care article” is meant an article, which in the ordinary course of usage, is used within the oral cavity.

By “oral care composition” is meant a composition, which in the ordinary course of usage, is not intentionally swallowed, but is rather retained in the oral cavity for a time sufficient to contact dental surfaces and/or oral tissues. The oral care composition may be in various forms including toothpaste, dentifrice, tooth gel, subgingival gel, mouth rinse, mousse, foam, mouthspray, lozenge, chewable tablet, chewing gum or denture care product. The oral care composition may also be incorporated onto strips or films for direct application or attachment to oral surfaces.

The term “teeth”, as used herein, refers to natural teeth as well as artificial teeth or dental prosthesis.

The term “open cell” as used herein, refers to a solid, interconnected, polymer-containing matrix that defines a network of spaces or cells that contain a gas, such as air.

The term “closed cell” as used herein, refers to a solid, polymer containing matrix that defines a network of spaces or cells that contain a gas, such as air, where the spaces or cell are not interconnected.

As used herein, the term “water-soluble polymer” is broad enough to include both water-soluble and water-dispersible polymers, and is defined as a polymer with a solubility in water, measured at 25° C., of at least about 0.1 gram/liter (g/L).

Active and other ingredients useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

### Oral Care Products and Methods

It is highly desirable to have oral care articles in the form of a flexible porous dissolvable solid structure, to be able to put a flexible porous dissolvable solid structure in an oral care article as an additive, or to be able to deliver a flexible porous dissolvable solid structure from a device. The use of such a structure as an article form allows for easy portability and the ability to better control dosing. For example, due to current restrictions on airlines regarding liquid products, a passenger is limited to carrying on only a small amount of mouthwash or to packing his mouthwash in his checked luggage. If the mouthwash was in a concentrated, single dose form, the passenger would be able to pack all that was needed into his carry-on without the need to worry about airline packing restrictions. The present inventors have discovered an at least partially open cell structure which allows not only for concentrated mouthwash, but many other types of oral care articles to be delivered in a flexible porous dissolvable solid structure form and which can be used within an oral care composition or delivered from a device.

### Flexible Porous Dissolvable Solid Structure Components

Open cell flexible porous dissolvable solid structures are generally made by first preparing a processing mixture. The processing mixture generally contains those components which will make up the dissolvable solid structure and a solvent. Some of the components which can make up the current open cell structure are listed below. The weight percentages listed below are by weight of the finished structure.

### Polymer Structurant

One component which is often included in an open celled porous dissolvable solid structure is a polymer structurant. Variations in the polymers used for the structurant can affect the final properties of the open celled structure and thereby control the dissolution and solubility behavior of the structure. For example, using a predominantly water soluble polymer structurant will result in a water soluble structure, while using a less water soluble or water insoluble polymer as the structurant will result in a reduced water solubility or even water insolubility.

The polymers for making the flexible porous dissolvable solid structures may be of synthetic or natural origin and may be modified by means of chemical reactions. In one embodiment, the polymer structurant is film forming. In another embodiment, the polymer structurant is a water-soluble polymer. In a further embodiment, the water soluble polymer structurant has a solubility in water from about 0.1 gram/liter (g/L) to about 500 grams/liter (g/L).

In one embodiment, the one or more polymers of the present invention are selected such that their weighted average molecular weight is from about 40,000 to about 500,000, in another embodiment from about 50,000 to about 400,000, in yet another embodiment from about 60,000 to about 300,000, and in still another embodiment from about 70,000 to about 200,000. The weighted average molecular weight is computed by summing the average molecular weights of each polymer raw material multiplied by their respective relative weight percentages by weight of the total weight of polymers present within the porous solid.

The water-soluble polymer may be present from about 10 wt % to about 70 wt % by weight of the solid structure, in one embodiment from about 15 wt % to about 40 wt %, and in a particular embodiment from about 20 wt % to about 30 wt % by weight of the structure.

Water-soluble polymer(s) of the present invention can include, but are not limited to, synthetic polymers including polyvinyl alcohols, polyvinylpyrrolidones, polyalkylene oxides, polyacrylates, caprolactams, polymethacrylates, polymethylmethacrylates, polyacrylamides, polymethylacrylamides, polydimethylacrylamides, polyethylene glycol monomethacrylates, polyurethanes, polycarboxylic acids, polyvinyl acetates, polyesters, polyamides, polyamines, polyethyleneimines, maleic/(acrylate or methacrylate) copolymers, copolymers of methylvinyl ether and of maleic anhydride, copolymers of vinyl acetate and crotonic acid, copolymers of vinylpyrrolidone and of caprolactam, vinyl pyrrolidone/vinyl acetate copolymers, copolymers of anionic, cationic and amphoteric monomers, and combinations thereof.

Suitable water-soluble polymer(s) may also be selected from naturally sourced polymers, including those of plant origin, examples of which include karaya gum, tragacanth gum, gum Arabic, acemannan, konjac mannan, acacia gum, gum ghatti, whey protein isolate, and soy protein isolate; seed extracts including guar gum, locust bean gum, quince seed, and psyllium seed; seaweed extracts such as Carrageenan, alginates, and agar; fruit extracts (pectins); those of microbial origin including xanthan gum, gellan gum, pullulan, hyaluronic acid, chondroitin sulfate, and dextran; and those of animal origin including casein, gelatin, keratin, keratin hydrolysates, sulfonic keratins, albumin, collagen, glutelin, glucagons, gluten, zein, and shellac.

Modified natural polymers are also useful as water-soluble polymers in the present invention. Suitable modified natural polymers include, but are not limited to, cellulose derivatives such as hydroxypropylmethylcellulose, hydroxymethylcellulose, hydroxyethylcellulose, methylcellulose, hydroxypropylcellulose, ethylcellulose, carboxymethylcellulose, cellulose acetate phthalate, nitrocellulose and other cellulose ethers/esters; and guar derivatives such as hydroxypropyl guar, and combinations thereof.

In one embodiment, the water soluble polymer is selected from the group consisting of: polyvinyl alcohols, polyvinylpyrrolidones, polyalkylene oxides, starch, starch derivatives, pullulan, gelatin, hydroxypropylmethylcelluloses, methycelluloses, carboxymethycelluloses, and combinations thereof. In a further embodiment, the water soluble polymer comprises polyvinyl alcohol, hydroxypropylmethylcellulose, or a combination thereof. Suitable polyvinyl alcohols include those available from Celanese Corporation (Dallas, Tex.) under the CELVOL® trade name. Suitable hydroxypropylmethylcelluloses include those available from the Dow Chemical Company (Midland, Mich.) under the METHOCEL® trade name including combinations with above mentioned hydroxypropylmethylcelluloses.

In a particular embodiment, the above mentioned water-soluble polymer(s) may be blended with any single starch or combination of starches as a filler material in such an amount as to reduce the overall level of water-soluble polymers required, so long as it helps provide the solid structure with the requisite structure and physical/chemical characteristics as described herein.

Typical sources for starch-based materials can include for example cereals, tubers, roots, legumes and fruits. Native sources can include corn, pea, potato, banana, barley, wheat, rice, sago, amaranth, tapioca, arrowroot, canna, sorghum, and waxy or high amylase varieties thereof. The starch-based materials may also include native starches that are modified using any modification known in the art, including physically modified starches, examples of which include sheared starches or thermally-inhibited starches; chemically modified starches including those which have been cross-linked, acetylated, and organically esterified, hydroxyethylated, and hydroxypropylated, phosphorylated, and inorganically esterified, cationic, anionic, nonionic, amphoteric and zwitterionic, and succinate and substituted succinate derivatives thereof; conversion products derived from any of the starches, including fluidity or thin-boiling starches prepared by oxidation, enzyme conversion, acid hydrolysis, heat or acid dextrinization, thermal and or sheared products may also be useful herein; and pregelatinized starches which are known in the art.

In one embodiment, the polymer structurant is hydrophobic. Hydrophobic polymers may include, but are not limited to the following families of polymers, poly alkyl acrylates, polydiene, poly imidazole, polylactone and polylactide, polyolefin, poly oxazoline, polyoxirane, polypyridine, polysiloxane, polystyrene, poly vinyl anthracene, poly vinyl naphthalene, poly(acrylonitrile), poly(adipic anhydride), polyester (ethylene terephthalate), polyesters (butylene bibenzoate), poly(ferrocenyldimethylsilane), poly(sulfone ether), poly(vinyl acetate) and poly(carbonate).

In one embodiment, the water insoluble polymers are selected from the group consisting of natural rosins such as wood rosins and gum rosins; vegetable proteins such as corn protein, pea protein or soy protein; hydrogenated castor oil; polyvinyl chloride; shellac; polyurethane; cellulose derivatives such as cellulose or ethylcellulose; waxes; polymers such as those sold under the Trade Mark Eudragit RS, and combinations thereof.

The polymer structurant may also include combinations of water soluble polymers, water insoluble polymers, or both

### Surfactants

In one embodiment, a flexible porous dissolvable solid structure of the present invention includes a surfactant. Surfactants suitable for use in the structure include anionic surfactants, nonionic surfactants, cationic surfactants, zwitterionic surfactants, amphoteric surfactants, or combinations thereof. The surfactant component may also include those that are intended primarily as a process aid in making a stable solid structure and thus, need not provide any lathering performance. Thus, the surfactant or combination of surfactants can either give a lathering or non-lathering article. Examples of such processing aid surfactants include mono- and di-glycerides, fatty alcohols, polyglycerol esters, propylene glycol esters, sorbitan esters and other emulsifiers known or otherwise commonly used to stabilize air interfaces. The surfactants may be present from about 1 wt % to about 70 wt % by weight of the structure, in one embodiment from about 2.5 wt % to about 60 wt %, in another embodiment from about 5 wt % to about 50 wt %, and in another embodiment from about 10 wt % to about 40 wt %.

Suitable anionic surfactants include those described in McCutcheon's Detergents and Emulsifiers, North American Edition (1986), Allured Publishing Corp.; McCutcheon's, Functional Materials, North American Edition (1992), Allured Publishing Corp.; and U.S. Pat. No. 3,929,678 (Laughlin et al.). Non-limiting examples of anionic surfactants suitable for use herein include alkyl and alkyl ether sulfates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, acyl glutamates, acyl sarcosinates, alkyl sulfoacetates, acylated peptides, alkyl ether carboxylates, acyl lactylates, anionic fluorosurfactants, sodium lauroyl glutamate, and combinations thereof.

In one embodiment, the alkyl and alkyl ether sulfates mentioned above have the respective formulae ROSO₃M and RO(C₂H₄O)ₓSO₃M, wherein R is alkyl or alkenyl of from about 8 to about 24 carbon atoms, x is 1 to 10, and M is a water-soluble cation such as ammonium, sodium, potassium and triethanolamine. In a further embodiment, the alkyl ether sulfates is made as a condensation product of ethylene oxide and monohydric alcohol's having from about 8 to about 24 carbon atoms. In one embodiment, R has from about 10 to about 18 carbon atoms in both the alkyl and alkyl ether sulfates. Sodium lauryl sulfate (SLS) and sodium coconut monoglyceride sulfonates are examples of anionic surfactant. Other suitable anionic surfactants include sarcosinates, such as sodium lauroyl sarcosinate, taurates, sodium lauryl sulfoacetate, sodium lauroyl isethionate, sodium laureth carboxylate, and sodium dodecyl benzenesulfonate. Combinations of anionic surfactants can also be employed. Many suitable anionic surfactants are disclosed by Agricola et al., U.S. Pat. No. 3,959,458.

Another class of anionic surfactants useful here are alkyl phosphates. The surface active organophosphate agents have a strong affinity for enamel surface and have sufficient surface binding propensity to desorb pellicle proteins and remain affixed to enamel surfaces. Suitable examples of organophosphate compounds include mono-, di- or triesters represented by the general structure below wherein Z₁, Z₂, or Z₃ may be identical or different, at least one being an organic moiety, in one embodiment selected from linear or branched, alkyl or alkenyl group of from 1 to 22 carbon atoms, optionally substituted by one or more phosphate groups; alkoxylated alkyl or alkenyl, (poly)saccharide, polyol or polyether group.

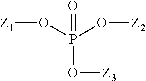

Some other agents include alkyl or alkenyl phosphate esters represented by the following structure:

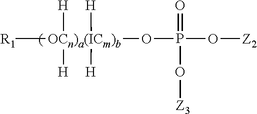

wherein R₁ represents a linear or branched, alkyl or alkenyl group of from 6 to 22 carbon atoms, optionally substituted by one or more phosphate groups; n and m, are individually and separately, 2 to 4, and a and b, individually and separately, are 0 to 20; Z₂ and Z₃ may be identical or different, each represents hydrogen, alkali metal, ammonium, protonated alkyl amine or protonated functional alkyl amine such as an alkanolamine, or a R₁—(OCₙH₂ₙ)ₐ(OCₘH₂ₘ)_{b}-group. Examples of suitable agents include alkyl and alkyl (poly)alkoxy phosphates such as lauryl phosphate; PPGS ceteareth-10 phosphate; Laureth-1 phosphate; Laureth-3 phosphate; Laureth-9 phosphate; Trilaureth-4 phosphate; C12-18 PEG 9 phosphate; Sodium dilaureth-10 phosphate. In one embodiment, the alkyl phosphate is polymeric. Examples of polymeric alkyl phosphates include those containing repeating alkoxy groups in the polymeric portion, in particular 3 or more ethoxy, propoxy, isopropoxy, or butoxy groups.

Additional suitable polymeric organophosphate agents include dextran phosphate, polyglucoside phosphate, alkyl polyglucoside phosphate, polyglyceryl phosphate, alkyl polyglyceryl phosphate, polyether phosphates and alkoxylated polyol phosphates. Some specific examples are PEG phosphate, PPG phosphate, alkyl PPG phosphate, PEG/PPG phosphate, alkyl PEG/PPG phosphate, PEG/PPG/PEG phosphate, dipropylene glycol phosphate, PEG glyceryl phosphate, PBG (polybutylene glycol) phosphate, PEG cyclodextrin phosphate, PEG sorbitan phosphate, PEG alkyl sorbitan phosphate, and PEG methyl glucoside phosphate. Suitable non-polymeric phosphates include alkyl mono glyceride phosphate, alkyl sorbitan phosphate, alkyl methyl glucoside phosphate, alkyl sucrose phosphates, and combinations thereof.

Another suitable surfactant is one selected from the group consisting of sarcosinate surfactants, isethionate surfactants and taurate surfactants. In one embodiment, an alkali metal or ammonium salts of these surfactants are used. Examples of those sodium and potassium salts include the following: lauroyl sarcosinate, myristoyl sarcosinate, palmitoyl sarcosinate, stearoyl sarcosinate and oleoyl sarcosinate, or combinations thereof.

Other suitable anionic surfactants include water-soluble salts of the organic, sulfuric acid reaction products of the general formula [R¹SO₃M], wherein R¹ is chosen from the group consisting of a straight or branched chain, saturated aliphatic hydrocarbon radical having from about 8 to about 24, in one embodiment from about 10 to about 18, carbon atoms; and M is a cation. In one embodiment, the anionic surfactant comprises an alkali metal or ammonium sulfonated C₁₀₋₁₈ n-paraffin.

Additional examples of suitable anionic surfactants are the reaction products of fatty acids esterified with isethionic acid and neutralized with sodium hydroxide where, for example, the fatty acids are derived from coconut oil; sodium or potassium salts of fatty acid amides of methyl tauride in which the fatty acids, for example, are derived from coconut oil. Other suitable anionic surfactants of this variety are described in U.S. Pat. No. 2,486,921, U.S. Pat. No. 2,486,922 and U.S. Pat. No. 2,396,278.

Still other suitable anionic surfactants are the succinamates, examples of which include disodium N-octadecylsulfosuccinamate; diammoniumlauryl sulfosuccinamate; tetrasodium N-(1,2-dicarboxyethyl)-N-octadecylsulfosuccinamate; diamyl ester of sodium sulfosuccinic acid; dihexyl ester of sodium sulfosuccinic acid; and dioctyl esters of sodium sulfosuccinic acid.

Other suitable anionic surfactants include olefin sulfonates having about 12 to about 24 carbon atoms. The α-olefins from which the olefin sulfonates are derived are mono-olefins having about 12 to about 24 carbon atoms, preferably about 14 to about 16 carbon atoms. Preferably, they are straight chain olefins.

Another class of anionic surfactants suitable for use herein is the β-alkyloxy alkane sulfonates. These compounds have the following formula:

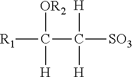

where R₁ is a straight chain alkyl group having from about 6 to about 20 carbon atoms, R₂ is a lower alkyl group having from about 1 to about 3 carbon atoms, and M is a water-soluble cation as hereinbefore described.

In one embodiment, the anionic surfactant comprises ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, monoethanolamine cocoyl sulfate, monoethanolamine lauryl sulfate, sodium tridecyl benzene sulfonate, sodium dodecyl benzene sulfonate, or a combination thereof. In varying embodiments, the present compositions comprise an anionic surfactant at a level of from about 1 wt % to about 70 wt % by weight of the structure, in one embodiment from about 2.5 wt % to about 60 wt %, in another embodiment from about 5 wt % to about 50 wt %, and in another embodiment from about 10 wt % to about 40 wt %.

Amphoteric surfactants suitable for use in the flexible porous dissolvable solid structures of the present invention include those that are broadly described as derivatives of aliphatic secondary and tertiary amines. In varying embodiments, the aliphatic radical can be straight or branched, one of the aliphatic substituents contains from about 8 to about 18 carbon atoms, and one contains an anionic water solubilizing group, e.g., carboxy, sulfonate, sulfate, phosphate, or phosphonate. Examples of these types of amphoteric surfactants include sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378.

Amphoteric surfactants suitable herein may also include alkylamphoacetates including lauroamphoacetate and cocoamphoacetate. Alkylamphoacetates can be comprised of monoacetates and diacetates.

Cationic surfactants can also be utilized. In one embodiment, the cationic surfactant is present at from about 0.1% to about 5% by weight. Cationic surfactants useful in the present invention include, for example, derivatives of quaternary ammonium compounds having one long alkyl chain containing from about 8 to 18 carbon atoms such as lauryl trimethylammonium chloride; cetyl pyridinium chloride; cetyl trimethylammonium bromide; coconut alkyltrimethylammonium nitrite; cetyl pyridinium fluoride or combinations thereof. Additional quaternary ammonium fluorides having detergent properties are described in U.S. Pat. No. 3,535,421 to Briner et al.

Suitable nonionic surfactants include those described in McCutcheon's Detergents and Emulsifiers, North American edition (1986), Allured Publishing Corp., and McCutcheon's Functional Materials, North American edition (1992). These nonionic surfactants suitable for use herein include, for example, alkyl glucosides, alkyl polyglucosides, polyhydroxy fatty acid amides, alkoxylated fatty acid esters, sucrose esters, amine oxides, and combinations thereof. Nonionic surfactants that can be used in the present invention include, for example, compounds produced by the condensation of alkylene oxide groups (hydrophilic in nature) with an organic hydrophobic compound which may be aliphatic or alkylaromatic in nature. Examples of suitable nonionic surfactants include the Pluronics® which are poloxamers, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and combinations of such materials.

Suitable zwitterionic surfactants include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Such suitable zwitterionic surfactants can be represented by the formula:

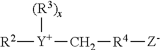

wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Other zwitterionic surfactants suitable for use herein include betaines, including high alkyl betaines such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl)sulfopropyl betaine and the like; amidobetaines and amidosulfobetaines, wherein the RCONH(CH₂)₃ radical, wherein R is a C₁₁-C₁₇ alkyl, is attached to the nitrogen atom of the betaine are also useful in this invention.

Other suitable surfactants are described in McCutcheon's, Emulsifiers and Detergents, 1989 Annual, published by M. C. Publishing Co., and in U.S. Pat. No. 3,929,678.

### Plasticizer

The flexible porous dissolvable solid structure may comprise a plasticizing agent. The plasticizing agent may be water soluble or water insoluble. Some non-limiting examples of suitable water soluble plasticizing agents include polyols, copolyols, polycarboxylic acids and salts, polyesters, glycerol esters, phthalic acid esters, fatty acids and esters, dimethicone copolyols, and combinations thereof.

Examples of useful polyols include, but are not limited to, glycerin, diglycerin, propylene glycol, ethylene glycol, butylene glycol, pentylene glycol, cyclohexane dimethanol, hexane diol, polyethylene glycol, sugar alcohols such as sorbitol, manitol, lactitol and other mono- and polyhydric low molecular weight alcohols (e.g., C₂-C₈ alcohols); mono di- and oligo-saccharides such as fructose, glucose, sucrose, maltose, lactose, and high fructose corn syrup solids, ascorbic acid, and combinations thereof.

Examples of polycarboxylic acids include, but are not limited to citric acid, maleic acid, succinic acid, polyacrylic acid, polymaleic acid, and combinations thereof. Examples of suitable polyesters include, but are not limited to, glycerol triacetate, acetylated-monoglyceride, diethyl phthalate, triethyl citrate, tributyl citrate, acetyl triethyl citrate, acetyl tributyl citrate, and combinations thereof. Examples of suitable dimethicone copolyols include, but are not limited to, PEG-12 dimethicone, PEG/PPG-18/18 dimethicone, PPG-12 dimethicone, and combinations thereof.

Other suitable water soluble plasticizers include, but are not limited to, alkyl and allyl phthalates; napthalates; lactates (e.g., sodium, ammonium and potassium salts); sorbeth-30; urea; lactic acid; sodium pyrrolidone carboxylic acid (PCA); sodium hyraluronate or hyaluronic acid; soluble collagen; modified protein; monosodium L-glutamate; alpha & beta hydroxyl acids such as glycolic acid, lactic acid, citric acid, maleic acid and salicylic acid; glyceryl polymethacrylate; polymeric plasticizers such as polyquaterniums; proteins and amino acids such as glutamic acid, aspartic acid, and lysine; hydrogen starch hydrolysates; other low molecular weight esters (e.g., esters of C₂-C₁₀ alcohols and acids); and mixtures thereof.

In one embodiment, the plasticizer comprises glycerin, propylene glycol, or a combination thereof. EP 0283165 B1 discloses other suitable plasticizers, including glycerol derivatives such as propoxylated glycerol.

In another embodiment, the plasticizer is water insoluble. Various kinds of organic powders and inorganic powders can be used as the water-insoluble plasticizer. Some examples of inorganic powders which are useful herein include, but are not limited to, microfine particles or granules of alumina, talc, magnesium stearate, titanium dioxide, barium titanate, magnesium titanate, calcium titanate, strontium titanate, zinc oxide, silica sand, clay, mica, tabular spar, diatomaceous earth, various inorganic oxide pigments, chromium oxide, cerium oxide, iron red, antimony trioxide, magnesium oxide, zirconium oxide, barium sulfate, barium carbonate, calcium carbonate, silica (colloidal or fumed), silicon carbide, silicon nitride, boron carbide, tungsten carbide, titanium carbide, carbon black, or mixtures thereof.

Some organic powders which are useful herein include, for example, cross-linked and non-cross-linked polymer powders, organic pigments, charge controlling agents, and waxes, for example. The cross-linked and non-cross-linked resin powders include, but are not limited to, resin powders of the styrene type, acrylic type, methacrylic type, polyethylene type, polypropylene type, silicone type, polyester type, polyurethane type, polyamide type, epoxy type, polyvinyl butyral type, rosin type, terpene type, phenol type, melamine type, and guanamine type, for example. Mixtures of any of the above organic or inorganic powders can also be used.

The plasticizer may be present from 0 wt % to about 25 wt %, by weight of the structure. In varying embodiments, the plasticizer is present in an amount from about 1 wt % to about 20 wt %, from about 3 wt % to about 15 wt %, or from about 5 wt % to about 10 wt %.

### Miscellaneous Ingredients

The flexible porous dissolvable solid structure may further comprise other ingredients. Other potential ingredients include organic solvents, especially water miscible solvents and co-solvents useful as solubilizing agents for polymeric structurants and as drying accelerators. Non-limiting examples of suitable solvents include alcohols, esters, ketones, aromatic hydrocarbons, aliphatic hydrocarbons, ethers, and combinations thereof. In one embodiment, the solvent comprises an alcohol or an ester. In a further embodiment, the alcohol is monohydric. In a still further embodiment, the monohydric alcohol is ethanol, iso-propanol, or n-propanol. In one embodiment, the ester is ethyl acetate or butyl acetate. Other non-limiting examples of suitable organic solvents are benzyl alcohol, amyl acetate, propyl acetate, acetone, heptane, iso-butyl acetate, iso-propyl acetate, toluene, methyl acetate, iso-butanol, n-amyl alcohol, n-butyl alcohol, hexane, methyl ethyl ketone, methanol, ethanol, n-propanol, isopropanol, n-butanol, isobutanol, methylethylketone, acetone, and combinations thereof.

Other preferred optional ingredients include latex or emulsion polymers, thickeners, clays, silicas, ethylene glycol distearate, deposition aids, including coacervate forming components and quaternary amine compounds.

### Oral Care Components

In varying embodiments, the flexible porous dissolvable solid structure includes an oral care component. Some oral care components are listed below. The structure comprises from about 0.5% to about 80%, by weight of the structure, of the oral care component. In one embodiment, the structure comprises from about 5% to about 60%, alternatively from about 10% to about 50%, by weight of the structure, of the oral care component.

### Carrier Material

One type of oral care component is a carrier material. Carrier materials can be either hydrophilic, hydrophobic, or a combination thereof. Examples of materials which can act as a carrier material include water, glycerin, sorbitol, polyethylene glycols, propylene glycol and other edible polyhydric alcohols, ethanol, hydrocarbons, natural vegetable oils, or combinations thereof.

### Flavors

Examples of some flavors and flavor components which are oral care components are mint oils, wintergreen, clove bud oil, cassia, sage, parsley oil, marjoram, lemon, orange, propenyl guaethol, heliotropine, 4-cis-heptenal, diacetyl, methyl-p-tert-butyl phenyl acetate, methyl salicylate, ethyl salicylate, 1-menthyl acetate, oxanone, a-irisone, methyl cinnamate, ethyl cinnamate, butyl cinnamate, ethyl butyrate, ethyl acetate, methyl anthranilate, iso-amyl acetate, iso-amyl butyrate, allyl caproate, eugenol, eucalyptol, thymol, cinnamic alcohol, octanol, octanal, decanol, decanal, phenylethyl alcohol, benzyl alcohol, α-terpineol, linalool, limonene, citral, neral, geranial, geraniol nerol, maltol, ethyl maltol, anethole, dihydroanethole, carvone, menthone, β-damascenone, ionone, γ-decalactone, γ-nonalactone, γ-undecalactone, or combinations thereof. Generally suitable flavoring ingredients include, for example, chemicals with structural features and functional groups that are less prone to redox reactions. These include derivatives of flavor chemicals that are saturated or contain stable aromatic rings or ester groups.

### Colorants

Examples of some colorants used in oral care compositions include D&C Yellow No. 10, FD&C Blue No. 1, FD&C Red No. 40, D&C Red No. 33 and combinations thereof.

### Sensates

Sensate molecules such as cooling, warming, and tingling agents are useful to deliver signals to the consumer. The most well-known cooling sensate compound is menthol, particularly l-menthol, which is found naturally in peppermint oil. Other isomers of menthol (neomenthol, isomenthol and neoisomenthol) have somewhat similar, but not identical odor and taste. The biggest difference among the isomers is in their cooling potency. L-menthol provides the most potent cooling.

Among synthetic coolants, many are derivatives of or are structurally related to menthol, i.e., containing the cyclohexane moiety, and derivatized with functional groups including carboxamide, ketal, ester, ether and alcohol. Examples include the ρ-menthanecarboxamide compounds such as N-ethyl-p-menthan-3-carboxamide. An example of a synthetic carboxamide coolant that is structurally unrelated to menthol is N,2,3-trimethyl-2-isopropylbutanamide. Additional examples of synthetic coolants include alcohol derivatives such as 3-1-menthoxypropane-1,2-diol, isopulegol, and ρ-menthane-3,8-diol; menthone glycerol acetal; menthyl esters such as menthyl acetate, menthyl acetoacetate, menthyl lactate, and monomenthyl succinate. Carboxamide cooling agents are described for example in U.S. Pat. Nos. 4,136,163; 4,150,052; 4,153,679; 4,157,384; 4,178,459 and 4,230,688.

Additional agents that are structurally unrelated to menthol but have been reported to have a similar physiological cooling effect include alpha-keto enamine derivatives described in U.S. Pat. No. 6,592,884 including 3-methyl-2-(1-pyrrolidinyl)-2-cyclopenten-1-one (3-MPC), 5-methyl-2-(1-pyrrolidinyl)-2-cyclopenten-1-one (5 -MPC), and 2,5-dimethyl-4-(1-pyrrolidinyl)-3(2H)-furanone (DMPF); icilin (also known as AG-3-5, chemical name 1-[2-hydroxyphenyl]-4-[2-nitrophenyl]-1,2,3,6-tetrahydropyrimidine-2-one) described in Wei et al., J. Pharm. Pharmacol. (1983), 35:110-112.

Some examples of warming sensates include ethanol; capsicum; nicotinate esters, such as benzyl nicotinate; polyhydric alcohols; capsicum powder; a capsicum tincture; capsicum extract; capsaicin; homocapsaicin; homodihydrocapsaicin; nonanoyl vanillyl amide; nonanoic acid vanillyl ether; vanillyl alcohol alkyl ether derivatives such as vanillyl ethyl ether, vanillyl butyl ether, vanillyl pentyl ether, and vanillyl hexyl ether; isovanillyl alcohol alkyl ethers; ethylvanillyl alcohol alkyl ethers; veratryl alcohol derivatives; substituted benzyl alcohol derivatives; substituted benzyl alcohol alkyl ethers; vanillin propylene glycol acetal; ethylvanillin propylene glycol acetal; ginger extract; ginger oil; gingerol; zingerone; or combinations thereof.

Examples of some tingling sensates include, jambu Oleoresin, Zanthoxylum peperitum, saanshool-I, saanshool II, sanshoamide, piperine, piperidine, eugenol, spilanthol, 4-(1-methoxymethyl)-2-phenyl-1,3-dioxolane, or combinations thereof.

### Sweeteners

Another component which can be included as an oral care component is a sweetener. Sweeteners can be both natural and artificial. Some suitable water-soluble sweeteners include monosaccharides, disaccharides and polysaccharides such as xylose, ribose, glucose (dextrose), mannose, galactose, fructose (levulose), sucrose (sugar), maltose, invert sugar (a combination of fructose and glucose derived from sucrose), partially hydrolyzed starch, corn syrup solids, dihydrochalcones, monellin, steviosides, glycyrrhizin, or combinations thereof. Suitable water-soluble artificial sweeteners include soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like. Other suitable sweeteners include dipeptide based sweeteners, such as L-aspartic acid derived sweeteners, such as L-aspartyl-L-phenylalanine methyl ester (aspartame), N-[N-(3,3-dimethylbutyl)-L-α-aspartyl]-L-phenylalanine 1-methyl ester (neotame), and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenyl-glycine, L-aspartyl-2,5-dihydro-L-phenylalanine, L-aspartyl-L-(1-cyclohexylen)-alanine, and the like. Water-soluble sweeteners derived from naturally occurring water-soluble sweeteners, such as a chlorinated derivative of ordinary sugar (sucrose), known, for example, under the product description of sucralose as well as protein based sweeteners such as thaumatoccous danielli (Thaumatin I and II) can be used.

### Actives

Some examples of actives as the oral care component include anticaries agents, antimicrobial agents, anti-inflammatory, antierosion, antistain, antisensitivity, antitartar agents, whitening agents, hydrating agents, bad breath reduction agents, and bleaching agents. Anticaries agents are generally used in an amount of about 0.01% to about 5.0%. It is common to have a fluoride compound present in dentifrices and other oral compositions in an amount sufficient to give a fluoride ion concentration in the composition of from about 0.0025% to about 5.0% by weight to provide anticaries effectiveness. In one embodiment, the fluoride concentration is from about 0.005% to about 2.0% by weight. A wide variety of fluoride ion-yielding materials can be employed as sources of soluble fluoride in the present compositions and methods. Examples of suitable fluoride ion-yielding materials are found in U.S. Pat. No. 3,535,421 to Briner et al. and U.S. Pat. No. 3,678,154 to Widder et al. Representative fluoride ion sources include: stannous fluoride, sodium fluoride, potassium fluoride, amine fluoride, sodium monofluorophosphate, indium fluoride, amine fluorides such as Olaflur, and many others.

Another type of active is an antimicrobial agent. One example of an antimicrobial agent is a quaternary ammonium compound and those useful herein include, for example, those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl-4-ethyl pyridinium chloride, dodecyl dimethyl(2-phenoxyethyl) ammonium bromide, benzyl dimethoylstearyl ammonium chloride, cetylpyridinium chloride, quaternized 5-amino-1,3-bis(2-ethyl-hexyl)-5-methyl hexahydropyrimidine, benzalkonium chloride, benzethonium chloride and methyl benzethonium chloride are exemplary of typical quaternary ammonium antibacterial agents. Other compounds include bis[4-(R-amino)-1-pyridinium]alkanes as disclosed in U.S. Pat. No. 4,206,215, Jun. 3, 1980 to Bailey. Other quaternary ammonium compounds include the pyridinium compounds. Examples of pyridinium quaternary ammonium compounds include cetylpyridinium and tetradecylpyridinium halide salts (i.e., chloride, bromide, fluoride and iodide). The quaternary ammonium antimicrobial agents can be included at levels of at least about 0.035%. In other embodiments they are included from about 0.045% to about 3.75% or from about 0.05% to about 1.0% by weight of the oral care component.

The present invention may also include other antimicrobial agents including non-cationic antimicrobial agents such as halogenated diphenyl ethers, phenolic compounds including phenol and its homologs, mono and poly-alkyl and aromatic halophenols, resorcinol and its derivatives, xylitol, bisphenolic compounds and halogenated salicylanilides, benzoic esters, and halogenated carbanilides. Also useful antimicrobials are enzymes, including endoglycosidase, papain, dextranase, mutanase, and combinations thereof. Such agents are disclosed in U.S. Pat. No. 2,946,725, Jul. 26, 1960, to Norris et al. and in U.S. Pat. No. 4,051,234 to Gieske et al. Examples of other antimicrobial agents include chlorhexidine, triclosan, triclosan monophosphate, and flavor oils such as thymol. Triclosan and other agents of this type are disclosed in Parran, Jr. et al., U.S. Pat. No. 5,015,466, and U.S. Pat. No. 4,894,220 to Nabi et al.

Another oral care component includes antitartar agents. One example of an antitartar agent is a polyphosphate. Polyphosphates have two or more phosphate units. An example of a polyphosphate antitartar agent is a pyrophosphate salt as a source of pyrophosphate ion. The pyrophosphate salts useful in the present compositions include, for example, the mono-, di- and tetraalkali metal pyrophosphate salts and combinations thereof. Disodium dihydrogen pyrophosphate (Na₂H₂P₂O₇), sodium acid pyrophosphate, tetrasodium pyrophosphate (Na₄P₂O₇), and tetrapotassium pyrophosphate (K₄P₂O₇) in their unhydrated as well as hydrated forms are further species. In articles of the present invention, the pyrophosphate salt may be present in one of three ways: predominately dissolved, predominately undissolved, or a combination of dissolved and undissolved pyrophosphate. The amount of pyrophosphate salt useful in making the oral care component is any tartar control effective amount. In varying embodiments, the amount of pyrophosphate salt is from about 0.1% to about 50%, from about 2% to about 10%, or about 3% to about 8%, by weight of the oral care component.

An additional example of an active is a bleaching agent. Bleaching agents are generally agents which whiten teeth. Examples of bleaching agents include peroxides, perborates, percarbonates, peroxyacids, persulfates, and combinations thereof. Suitable peroxide compounds include, for example, hydrogen peroxide, urea peroxide, calcium peroxide, sodium peroxide, zinc peroxide, or combinations thereof. One example of a percarbonate is sodium percarbonate. An example of a persulfate includes oxones. The following amounts represent the amount of peroxide raw material, although the peroxide source may contain ingredients other than the peroxide raw material. For example, the peroxide source could be a solution a peroxide raw material and a carrier material. Generally, the present composition may contain from about 0.01% to about 30% of peroxide raw material. In other embodiments, the peroxide raw material is from about 0.1% to about 10% or from about 0.5% to about 5%, by weight of the oral care component.

Another active is a bad breath reduction agent. These agents generally work to reduce breath malodor. Examples of bad breath reduction agents include copper salts and carbonyl compounds such as ascorbic acid [3-oxo-L-gulofuranolactone]; cis-jasmone [3-methyl-2-(2-pentenyl-2-cyclopentenone]; 2,5-dimethyl-4-hydroxy-3(2H)-furanone; 5-ethyl-3-hydroxy-4-methyl-2(5H)-furanone; vanillin[4-hydroxy-3-methoxybenzaldehyde]; ethyl vanillin; anisaldehyde[4-methoxybenzaldehyde]; 3,4-methylenedioxybenzaldehyde; 3,4-dimethoxybenzaldehyde; 4-hydroxybenzaldehyde; 2-methoxybenzaldehyde; benzaldehyde; cinnamaldehyde[3-phenyl-2-propenal]; hexyl cinnamaldehyde; α-methyl cinnamaldehyde; ortho-methoxy cinnamaldehyde; or combinations thereof. Without being limited by theory, it is believed some bad breath reduction agents work as “traps” by reacting with the thiol or sulfide and forming products with less odor impact.

Additional active agents include those that can be delivered systemically through the oral cavity.

### Metal Salts

Metal salts have a wide range of functions from antimicrobial agents to sensitivity agents and/or buffers. In one embodiment, the metal salt comprises a zinc salt, stannous salt, potassium salt, aluminum salt, calcium salt, copper salt, or a combination thereof. In a further embodiment, the zinc salt is selected from the group consisting of zinc fluoride, zinc chloride, zinc iodide, zinc chlorofluoride, zinc actetate, zinc hexafluorozirconate, zinc sulfate, zinc lactate, zinc tartrate, zinc gluconate, zinc citrate, zinc malate, zinc glycinate, zinc pyrophosphate, zinc metaphosphate, zinc oxalate, zinc phosphate, zinc carbonate, and combinations thereof. In another embodiment, the zinc salt comprises zinc chloride, zinc citrate, zinc gluconate, zinc lactate, zinc oxide, or combinations thereof.

In an additional embodiment, the potassium salt is selected from the group consisting of potassium nitrate, potassium citrate, potassium oxalate, potassium bicarbonate, potassium acetate, potassium chloride, and combinations thereof. In a further embodiment, the potassium salt comprises potassium nitrate, potassium citrate, potassium chloride, or combinations thereof.

In an additional embodiment, the copper salt is selected from the group consisting of copper fluoride, copper chloride, copper iodide, copper chlorofluoride, copper actetate, copper hexafluorozirconate, copper sulfate, copper lactate, copper tartrate, copper gluconate, copper citrate, copper malate, copper glycinate, copper pyrophosphate, copper metaphosphate, copper oxalate, copper phosphate, copper carbonate, and combinations thereof. In a further embodiment, the copper salt comprises copper gluconate, copper acetate, copper glycinate, or a combination thereof.

In another embodiment, the stannous salt is selected from the group consisting of stannous fluoride, stannous chloride, stannous iodide, stannous chlorofluoride, stannous actetate, stannous hexafluorozirconate, stannous sulfate, stannous lactate, stannous tartrate, stannous gluconate, stannous citrate, stannous malate, stannous glycinate, stannous pyrophosphate, stannous metaphosphate, stannous oxalate, stannous phosphate, stannous carbonate, stannous gluconate, and combinations thereof. In a further embodiment, the stannous salt comprises stannous fluoride, stannous chloride, stannous chloride dihydrate, stannous fluoride, stannous lactate, stannous gluconate, stannous sulfate, or a combination thereof.

Dentifrices containing stannous salts, particularly stannous fluoride and stannous chloride, are described in U.S. Pat. No. 5,004,597 to Majeti et al. Other descriptions of stannous salts are found in U.S. Pat. No. 5,578,293 issued to Prencipe et al. and in U.S. Pat. No. 5,281,410 issued to Lukacovic et al. In addition to the stannous ion source, other ingredients needed to stabilize the stannous may be included, such as the ingredients described in Majeti et al. and Prencipe et al.

The metal salt will be present in an amount to deliver metal ions from about 0.05% to about 11%, by weight of the oral care composition in one embodiment. In other embodiments, the metal ions are present in an amount of from about 0.5 to about 7% or from about 1% to about 5%. In additional embodiments, the stannous ions are present in an amount of from about 0.1 to about 7% or from about 1% to about 5% or from about 1.5% to about 3% by weight of the oral care composition. In certain embodiments, the amount of zinc or copper ions used in the present invention can range from about 0.01 to about 5%. In other embodiments the amount of zinc or copper ions are from about 0.05 to about 4% or from about 0.1 to about 3.0%.

### Miscellaneous Oral Care Components

In addition to the above, other components may be included as oral care components to achieve the desired benefit. These miscellaneous components include, for example, chelating agents, abrasives, salivation agents, fillers, solvents, emollients, refractive particles (ex. mica), thickeners, buffers, humectants, binders, opacifiers, disintegrants, diluents, lubricants, adhesives, and extracts of natural components.

### Physical Characteristics

Flexible porous dissolvable solid structures may be formed by trapping a gas within a liquid or solid. Solid structures can be classified into two types based on their pore structure. The first type is called open cell structure. This structure has pores that are connected and form an interconnected network which is relatively soft. The second type of structure, closed cell, does not have interconnected pores. Normally the closed cell structures have higher compressive strength due to their structures and can be used as insulators when filled with air. However, the open cell structure will fill with whatever surrounds it.

The solid strucures of the present invention have a predominantly open celled structure which allows for them to have varying properties based at least partly on the open cell structure.

### Percent Open Cell Content

One way to measure the open structure of a flexible porous dissolvable solid strucutre is through its percent open cell content. In one embodiment, the percent open cell content is measured via gas pycnometry. Gas pycnometry is a common analytical technique that uses a gas displacement method to measure volume accurately. Inert gases, such as helium or nitrogen, are used as the displacement medium. The sample is sealed in the instrument compartment of known volume, the appropriate inert gas is admitted, and then expanded into another precision internal volume. The pressure before and after expansion is measured and used to compute the sample volume. Dividing this volume into the sample weight gives the gas displacement density. ASTM Standard Test Method D2856 provides a procedure for determining the percentage of open cells using an older model of an air comparison pycnometer. This device is no longer manufactured. However, you can determine the percentage of open cells conveniently and with precision by performing a test which uses Micromeritics' AccuPyc Pycnometer. The ASTM procedure D2856 describes 5 methods (A, B, C, D, and E) for determining the percent of open cells of porous solid structure materials. In one embodiment, method C of the ASTM procedure is used to calculate to percent open cells. This method simply compares the geometric volume as determined using calipers and standard volume calculations to the true volume as measured by the Accupyc. The samples can be analyzed using an Accupyc 1340 using nitrogen gas with the ASTM foampyc software. More information on this technique is available on the Micromeritics Analytical Services web sites (www.particletesting.com or www.micromeritics.com).

The percent open cell, like the polymer structurant, can contribute to the dissolution properties of the structure. For example, a structure having a hydrophilic polymer structurant and an open cell percentage from about 80% to 100% will have a high dissolution rate. In another embodiment, a structure with a slower dissolution rate can be used by lowering the open cell percent to below 80% and/or changing the polymer structurant being used. In a preferred embodiment, the structure has a percent open-cell content of from about 80% to about 100%, in one embodiment from about 85% to about 97.5%, and in another embodiment from about 90% to about 95%.

### Dissolution Rate

The flexible porous dissolvable solid structure has a dissolution rate which allows the structure to operate within the desired parameters for the end use. Thus, in one embodiment the structure can rapidly disintegrate during use, while in another embodiment, the structure will disintegrate more gradually and/or only to a small degree. In one embodiment, the dissolution rate of the structure may be determined in accordance with the following Conductivity Dissolution Method.

Conductivity Dissolution Method: In a 250 ml beaker, 150+/−0.5 grams of distilled water is weighed at room temperature (25° C.). The beaker is placed on an orbital shaker, for example a VWR model DS-500E and started at 150 RPM. A conductivity probe, for example a VWR model 2052 connected to a VWR conductivity meter, is submerged just below the surface of the water in such a manner that the conductivity probe remains stationary in relation to the motion of the beaker and never touches the side of the beaker. A 0.20+/−0.01 grams of the structure is weighed and placed into the water. Conductivity data is recorded every 15 seconds for 6 minutes, and then once a minute until 30 minutes. The final value is recorded when the conductivity values stopped changing or 30 minutes is reached, whichever is earlier. The conductivity dissolution time is taken as the time it takes in seconds until the conductivity values stop changing or as the maximum of 30 minutes, which ever happens first. For those products which have a solubility time of less than 15 seconds or greater than 30 minutes, the method can be adjusted accordingly.

In one embodiment, the structure has a conductivity dissolution time of from about 100 seconds to about 1,200 seconds, in another embodiment from about 110 seconds to about 900 seconds, in yet another embodiment from about 120 seconds to about 600 seconds, and in still another embodiment from about 130 seconds to about 300 seconds. In another embodiment, the conductivity dissolution time is greater than 1200 seconds. The more the intended use requires a sustained delivery, the longer the conductivity time will need to be.

### Wall Thickness

The walls of the flexible porous dissolvable solid structure will also have a wall thickness. In varying embodiments, the wall thickness is from about 0.02 mm to about 0.15 mm, in one embodiment from about 0.025 mm to about 0.12 mm, in another embodiment from about 0.03 mm to about 0.09 mm, and in still another embodiment from about 0.035 mm to about 0.06 mm In one embodiment, cell wall thickness is computed from scanned images via a micro computed tomography system (μCT80, SN 06071200, Scanco Medical AG) as described herein. As used herein, the “Cell Wall Thickness” is determined according to the method defined for the measurement of Trabecular Thickness using Scanco Medical's Bone Trabecular Morphometry evaluation. The definition of Trabecular Thickness as taken from the Scanco User's manual: Trabecular Thickness uses a Euclidean distance transformation (EDM), which calculates the Euclidean distance from any point in the foreground to the nearest background point. The Trabecular Thickness measure represents twice the centerline values associated with the local maxima of the EDM, which represents the distance to the center of the object (twice this distance will yield the thickness).

### Specific Surface Area

The flexible porous dissolvable solid structure also has a specific surface area. In varying embodiments, the structure has a specific surface area of from about 0.01 m²/g to about 0.25 m²/g, from about 0.015 m²/g to about 0.22 m²/g, from about 0.04 m²/g to about 0.19 m²/g, and from about 0.045 m²/g to about 0.16 m²/g. The specific surface area is measured via a gas adsorption technique. Surface Area is a measure of the exposed surface of a solid sample on the molecular scale. The BET (Brunauer, Emmet, and Teller) theory is the most popular model used to determine the surface area and is based upon gas adsorption isotherms. Gas Adsorption uses physical adsorption and capillary condensation to measure a gas adsorption isotherm. The technique is summarized by the following steps; a sample is placed in a sample tube and is heated under vacuum or flowing gas to remove contamination on the surface of the sample. The sample weight is obtained by subtracting the empty sample tube weight from the combined weight of the degassed sample and the sample tube. The sample tube is then placed on the analysis port and the analysis is started. The first step in the analysis process is to evacuate the sample tube, followed by a measurement of the free space volume in the sample tube using helium gas at liquid nitrogen temperatures. The sample is then evacuated a second time to remove the helium gas. The instrument then begins collecting the adsorption isotherm by dosing krypton gas at user specified intervals until the requested pressure measurements are achieved. Samples may then analyzed using an ASAP 2420 with krypton gas adsorption.

### Basis Weight

The flexible porous dissolvable solid structure can have a basis weight. In varying embodiments the structure has a basis weight 125 grams/m² to about 1,500 grams/m², from about 150 grams/m² to about 1,200 grams/m², from about 200 grams/m² to about 1,000 grams/m², and from about 300 grams/m² to about 800 grams/m². The basis weight is calculated as the weight of the porous solid component per area of the selected porous solid (grams/m²). The area is calculated as the projected area onto a flat surface perpendicular to the outer edges of the porous solid. For a flat object, the area is thus computed based on the area enclosed within the outer perimeter of the sample. For a spherical object, the area is thus computed based on the average diameter as 3.14×(diameter/2)². For a cylindrical object, the area is thus computed based on the average diameter and average length as diameter×length. For an irregularly shaped three dimensional object, the area is computed based on the side with the largest outer dimensions projected onto a flat surface oriented perpendicularly to this side. This can be accomplished by carefully tracing the outer dimensions of the object onto a piece of graph paper with a pencil and then computing the area by approximate counting of the squares and multiplying by the known area of the squares or by taking a picture of the traced area (preferably shaded-in for contrast) including a scale and using image analysis techniques.

### Dry Density

The flexible porous dissolvable solid structure can have a dry density. In varying embodiments, the structure has a dry density of from about 0.03 g/cm³ to about 0.5 g/cm³, from about 0.04 g/cm³ to about 0.3 g/cm³, from about 0.05 g/cm³ to about 0.2 g/cm³, and from about 0.06 g/cm³ to about 0.15 g/cm³. The dry density of the porous solid is determined by the equation: Calculated Density=Basis Weight of porous solid/(Porous Solid Thickness×1,000). The Basis Weight and Thickness of the porous solid are determined in accordance with the methodologies described herein.

### Product Form

The flexible porous dissolvable solid structure can be produced in a variety of forms. In one embodiment, the structure is in the form of a predominantly open celled porous solid. The predominantly open celled porous solid can have a range of solubility. For example, the porous solid may be water soluble, partially water soluble, or water insoluble. The amount and type of solubility can be determined based on the product form and intended use.

The structure can, for example, be in the form of one or more flat sheets or pads. The pad may be in any suitable shape. The pads can also be in the form of a continuous strip which could, for example, be delivered in a tape-like roll dispenser with individual portions dispensed via perforations and or a cutting mechanism. The structure could also have a more rounded or cylindrical shape. The structure can also be of any suitable size. The structure, for example, could be of a size and shape to fit on a toothbrush, to be used as an insert into device, or it could be of a very small size such that it could be added to an oral care composition like toothpaste.

The structure may comprise one or more textured, dimpled or otherwise topographically patterned surfaces including letters, logos or figures. Additionally, the structure can be perforated with holes or channels penetrating into or through the porous solid. These perforations can be formed, for example, during the drying process via spikes extended from the surface of the underlying mold, belt or other non-stick surface. Alternatively, these perforations can be formed after the drying process via poking or sticking the porous solids with pins, needles or other sharp objects. It has been found that such perforations can increase the dissolution rate of the structure relative to un-perforated structure.

In another embodiment the structure can be designed to comprise multiple layers. Each layer may contain different ingredients, different properties, same ingredients, same properties, or a combination thereof. For example different layers may have different dissolution rates or solubility. In another embodiment the structure may have a water insoluble backing or layer.

### Surface Resident Coatings Comprising Oral Care Component

In one embodiment, the open-celled flexible porous dissolvable solid structures of the present invention provide a continuous and accessible high surface area “scaffold” (a 3-D fibrous web network) which can serve as a delivery system for physically adsorbed oral care component(s) and oral care component complexes present as surface resident coatings onto the scaffold. This location puts the coating in position to immediately contact water during use for the release of the oral care component(s) and enables significant flexibility during manufacturing by enabling “late stage differentiation” whereby differing product versions and compositions can be directly prepared building from the same fibrous substrate composition and after the fibrous substrate is produced.

In one embodiment the ratio of the porous solid structure to the surface resident coating comprising said at least one oral care component is from about 110:1 to about 0.1:1, in another embodiment from about 20:1 to about 0.2:1, and in another embodiment from about 10:1 to about 0.3:1, and in yet another embodiment from about 1:1 to about 0.4:1.

The surface resident coating of the present invention comprises one or more oral care components as defined herein. Those of skill in the art will understand that the porous solid structure can also include one or more oral care components that are blended or otherwise combined together within a single surface resident coating or that may be applied via a multiplicity of different surface resident coatings that may or may not be in contact with one another (applied as as layers or to differing regions of the structure or combinations thereof) and wherein each surface resident coating may comprise the same or a different composition and the same or different physical form than the other surface resident coatings.

In one embodiment, the amount of surface resident coating can be from about 1% to about 70%, by weight of the oral care article, of a coating oral care component at least partially coating said structure, alternatively from about 10%, or from about 25%, or from about 40%, up to about 60%, or up to about 50% of said oral care component.

In one embodiment, the porous solid structure comprises an oral care component which can be the same or different from said oral care component present in the coating (the coating oral care component). In one embodiment, the surface resident oral care component is one or more surfactants in addition to the at least one surfactant already present in the substrate.

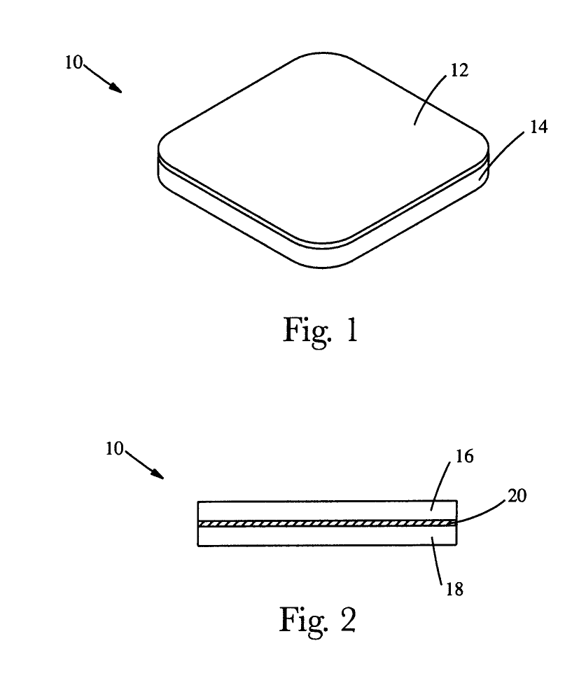

The surface resident coating of the present invention may be applied to the porous solid structure. In one embodiment, the surface resident coating is in the form of a fine powder. As seen in FIG. 1, in certain embodiments of the present invention, the oral care article 10 contains a surface resident coating 12 that is located on at least a portion of the surface of the structure 14. It will be appreciated that the surface resident coating 12 may not always be adjacent to the structure 14. In certain embodiments, the surface resident coating 12 may permeate the structure 14 in whole or in part.

Alternatively, the surface resident coating can be included (e.g., sandwiched or encased) within the oral care article or parts thereof. Such a surface resident coating can be sprayed, dusted, sprinkled, coated, surface-printed (e.g., in the shape of a desired adornment, decoration, or pattern), poured on, injected into the interior, dipped, or by any other suitable means, such as by use of a depositor, sifter, or powder bed.

Those of skill in the art should understand that the coating can be applied as a powder coating or can be a fluid coating. For instance, where the coating is a fluid coating, the coating can be sprayed, spread, dropped, printed, sandwiched between different articles or different portions of the same article, layered, injected, rolled on, or dipped. The coating can be applied over portions or entire regions of the article's exterior surface, and can be applied in a manner to adorn, decorate, form a logo, design, etc.

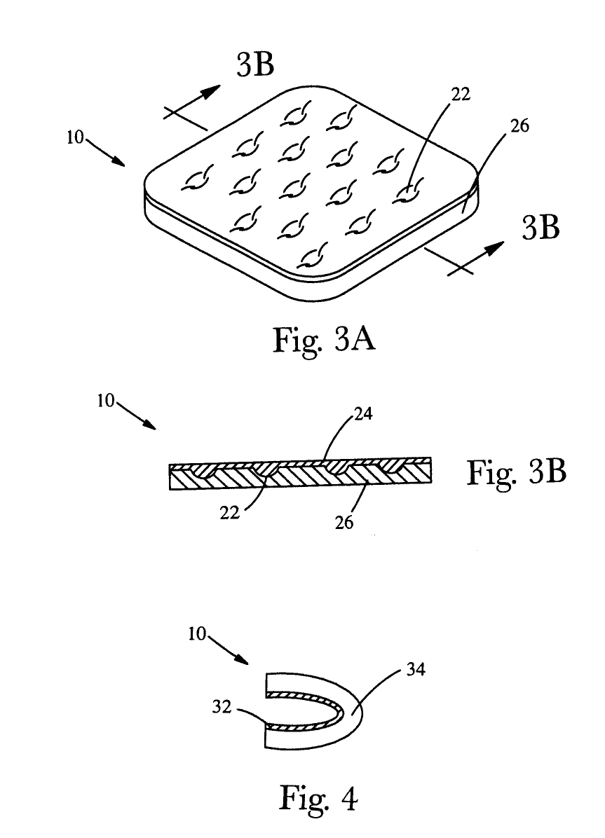

In the embodiments depicted by FIGS. 3A, 3B, and 4, the oral care article 10 contains a surface resident coating that can be situated below the surface of the porous solid structure. As seen in FIG. 3B which is a cross sectional view of the oral care article 10, the surface resident coating 24 is located within the dimples 22 of the structure 26.

Referring now to FIG. 2, in certain embodiments the powder is sandwiched between two porous solid structures which are then joined together (e.g., via sealing the adjoining surfaces or edges with a thin layer of water and/or plasticizer so as to not substantially dissolve the structure and applied pressure to induce adhesion). In this embodiment, the oral care article 10 comprises two porous solid structures 16, 18 in between which a surface resident coating 20 is located. In another embodiment, the substrate is at least partially coated with a first coating and a second coating, wherein the multiple coatings can be applied to separate areas of the substrate, such as separate sides of the substrate, or the multiple coatings can be applied one over the other.

Alternatively, in certain embodiments, the powder may be on one oral care article which is folded over to form a pouch, encasing the powder. As depicted in FIG. 4, the oral care article 10 comprises a surface resident coating 32 that is enclosed within a folded structure 34.

The oral care article may comprise one or more textured, dimpled or otherwise topographically patterned surfaces including letters, logos or figures. The textured substrate can result from the shape of the substrate, in that the outermost surface of the substrate contains portions that are raised with respect to other areas of the surface. The raised portions can result from the formed shape of the oral care article, for example the oral care article can be formed originally in a dimpled or waffle pattern. The raised portions can also be the result of creping processes, imprinted coatings, embossing patterns, laminating to other layers having raised portions, or the result of the physical form of the structure itself. The texturing can also be the result of laminating one structure to a second structure that is textured. In a particular embodiment, the oral care article can be perforated with holes or channels penetrating into or through the fibrous solid.

### Water-Releasible Matrix Complexes Comprising Oral Care Components

In one embodiment, the oral care articles of the present invention encompass one or more water-releasable matrices comprising oral care components. The one or more water-releasible matrix complexes comprising oral care components may be incorporated into the composition that is manipulated to form the flexible porous dissolvable solid structure. In a preferred embodiment, the water-releasible matrix complexes comprising oral care components are incorporated within the surface resident coatings as defined herein. In one embodiment the surface resident coating comprises from about 10% to about 100% of one or more water-releasible matrix complexes comprising oral care components, in another embodiment from about 25% to about 100%, and in yet another embodiment from about 40% to about 100%.

The ratio of the water-releasible matrix material to the one or more oral care components in the complex is in one embodiment from about 0.5:1 to about 19:1, in another embodiment from about 0.7:1 to about 6:1, and in yet another embodiment from about 1:1 to about 3:1. The water-releasible matrix complexes comprising oral care components according to the invention are in particulate form and may have a particle size from about 1 μm to about 200 μm, in another embodiment from about 2 μm to about 100 μm, and in yet another embodiment from about 3 μm to about 50 μm.

The water-releasible matrix materials of the present invention may include cyclodextrins, as well as high surface area particles that form complexes such as starches, polyethylenes, polyamides, polystyrenes, polyisoprenes, polycarbonates, polyesters, polyacrylates, vinyl polymers polyurethanes, amorphous silica, amorphous silica gel, precipitated silica, fumed silica, aluminosilicates, such as zeolites and alumina, silicates, carbonates, and mixtures thereof. Preferred water-releasible matrix materials include cyclodextrin complexes, silicates, silicas, carbonates, and starch-based materials.

Cyclodextrin water-releasible matrix materials include unsubstituted cyclodextrins containing from about six to about twelve glucose units, especially alpha-cyclodextrin, beta-cyclodextrin, gamma-cyclodextrin and/or their derivatives and/or mixtures thereof. For example, the present invention may use cyclodextrins selected from the group consisting of beta-cyclodextrin, hydroxypropyl alpha-cyclodextrin, hydroxypropyl beta-cyclodextrin, methylated-alpha-cyclodextrin, methylated-beta-cyclodextrin, and mixtures thereof. A more complete description of the cyclodextrins, cyclodextrin derivatives and cyclodextrin particle sizes useful in the matrices of the present invention may be found in U.S. Pat. No. 5,429,628, issued to Trinh et al. on Jul. 4, 1995. A preferred cyclodextrin water-releasible matrix material is beta-cyclodextrin.

Inorganic water-releasible matrix materials include silicas (silicon dioxide), silicates or carbonates wherein the silicates and carbonates are formed by reaction of a carbonate or silicate with the alkali (IA) metals, alkaline earth (IIA) metals, or transition metals. Inorganic materials suitable for use herein include calcium silicate, amorphous silicas, calcium carbonate, magnesium carbonate, sodium aluminosilicate, or zinc carbonate, and mixtures thereof. Some specific examples of the silicates and carbonates useful in the present invention are more fully explained in Van Nostrand Reinhold's Encyclopedia of Chemistry, 4th Ed. pages 155, 169, 556, and 849, (1984), which is incorporated herein by reference. Synthetic versions of the inorganic materials exist, particularly in regards to silicas and silicates. Synthetic versions are formed by controlled chemical reactions in a manufacturing process rather than using a natural, mined version of these compounds which is then further refined. Synthetic carbonates useful in the present invention can be obtained from various suppliers such as Mallinckrodt or Whittaker, Clark, and Daniels. Examples of synthetic calcium silicates useful in the present invention are Hubersorb® 250, or Hubersorb® 600 available from J. M. Huber (Havre de Grace, Md.). Examples of synthetic silicon dioxides useful in the present invention are Zeofree® 80, Zeosyl® 110SD, Zeosyl 200, Zeofree 5161, Zeofree 5162 and Zeothix® 265 also available from J. M. Huber. Examples of synthetic sodium aluminosilicates useful in the present invention include Zeolex® 7, Zeolex 201, Zeolex 23A and Zeolex 7A also available from J. M. Huber. Inorganic materials suitable for use in the present invention preferably include synthetic calcium silicates. In one embodiment the synthetic calcium silicate of the present invention is Hubersorb® 600 from J. M. Huber which is reported to have an oil absorption of 475 cc/100 g, an average particle size of 6 microns, a BET surface area of 300 square meters per gram and a bulk density of 8 lbs/CFT.

Starch-based water-releasible matrix materials include native starches that are modified using any modification known in the art, including physically modified starches examples of which include sheared starches or thermally-inhibited starches; chemically modified starches including those which have been cross-linked, acetylated, and organically esterified, hydroxyethylated, and hydroxypropylated, phosphorylated, and inorganically esterified, cationic, anionic, nonionic, amphoteric and zwitterionic, and succinate and substituted succinate derivatives thereof; conversion products derived from any of the starches, including fluidity or thin-boiling starches prepared by oxidation, enzyme conversion, acid hydrolysis, heat or acid dextrinization, thermal and or sheared products may also be useful herein; and pregelatinized starches which are known in the art. Starch based materials suitable for use herein include acid modified starches, enzymatic hydrolyzed starches, octenyl succinic acid anhydride modified starches (OSAN starches), dextrinized OSAN starches, dextrins, maltodextrins, pregelatinized waxy maize starches, and mixtures thereof. Suitable examples of such starch based materials include, but are not limited to, CAPSUL™, CAPSUL TA™, HI-CAP 100™, CAPSUL E™, NARLEX™ (ST and ST2), AND N-LOK™, manufactured by Akzo Nobel (Bridgewater, N.J.); the EmCap™ series including 12633, 12634, 12635, 12639, 12635, and 12671, manufactured by Cargill Inc. (Cedar Rapids, Iowa); and STA-DEX® 90 and MIRA-CAP® Starch, manufactured by Tate & Lyle (Decatur, Ill.). Other examples of modified starches suitable for the present invention are disclosed for example in WO 99/55819, WO 01/40430, EP-A-858828, EP-A-1160311 and U.S. Pat. No. 5,955,419.

### Microcapsules Comprising Oral Care Components

In an additional embodiment, the oral care articles of the present invention encompass one or more microcapsules comprising oral care components. The one or more microcapsules comprising oral care components may be incorporated into the composition that is manipulated to form the flexible porous dissolvable solid structure. In one embodiment, the microcapsules comprising oral care components are incorporated within the surface resident coatings as defined herein. In one embodiment the surface resident coating comprises from about 10% to about 100% of one or more microcapsules comprising oral care components, in another embodiment from about 25% to about 100%, and in yet another embodiment from about 40% to about 100%. For purposes of the present invention and unless indicated otherwise, the terms “perfume nanocapsule” and “microcapsule” are within the scope of the term “perfume microcapsule.”

The microcapsules may be formed by a variety of procedures that include, but are not limited to, coating, extrusion, spray-drying, interfacial, in-situ and matrix polymerization. The possible shell materials vary widely in their stability toward water. Among the most stable are polyoxymethyleneurea (PMU)-based materials, which may hold certain oral care components for even long periods of time in aqueous solution (or product). Such systems include but are not limited to urea-formaldehyde and/or melamine-formaldehyde. Gelatin-based microcapsules may be prepared so that they dissolve quickly or slowly in water, depending for example on the degree of cross-linking. Many other capsule wall materials are available and vary in the degree of oral care component diffusion stability observed. Without wishing to be bound by theory, the rate of release of oral care component from a capsule, for example, once deposited on a surface is typically in reverse order of in-product oral care component diffusion stability. As such, urea-formaldehyde and melamine-formaldehyde microcapsules for example, typically require a release mechanism other than, or in addition to, diffusion for release, such as mechanical force (e.g., friction, pressure, shear stress) that serves to break the capsule and increase the rate of oral care component release. Other triggers include melting, dissolution, hydrolysis or other chemical reaction, electromagnetic radiation, and the like. The use of pre-loaded microcapsules requires the proper ratio of in-product stability and in-use and/or on-surface (on-situs) release, as well as proper selection of oral care components. Microcapsules that are based on urea-formaldehyde and/or melamine-formaldehyde are relatively stable, especially in near neutral aqueous-based solutions. These materials may require a friction trigger which may not be applicable to all product applications. Other microcapsule materials (e.g., gelatin) may be unstable in aqueous-based products and may even provide reduced benefit (versus free perfume control) when in-product aged. Scratch and sniff technologies are yet another example of microcapsule aided delivery of oral care components. Suitable microcapsules may include those described in the following references: U.S. Patent Application Nos.: 2003/0125222 A1; 2003/215417 A1; 2003/216488 A1; 2003/158344 A1; 2003/165692 A1; 2004/071742 A1; 2004/071746 A1; 2004/072719 A1; 2004/072720 A1; 2006/0039934 A1; 2003/203829 A1; 2003/195133 A1; 2004/087477 A1; 2004/0106536 A1; and U.S. Patent Nos.: 6,645,479 B1; 6,200,949 B1; 4,882,220; 4,917,920; 4,514,461; 6,106,875 and 4,234,627, 3,594,328 and U.S. RE Pat. No. 32713.

### Products Types and Associated Methods of Use

Non-limiting examples of product type embodiments for this invention include, for example, concentrated mouth rinse, concentrated toothpaste, solid dentifrice, denture adhesive, vehicle for topical active delivery (e.x. tooth whitening product), vehicle for systemic active delivery (e.x. wafer, lozenge, or chew), easily hydrated binders for manufacturing of oral care compositions, as an additive in an oral care composition, etc.

In one embodiment, the flexible porous dissolvable solid structure is an oral care article in the form of a concentrated mouthwash. In one embodiment, the concentrated mouthwash would be in the form of a dissolvable wafer. In a further embodiment, a method of using a concentrated mouthwash would include, adding the concentrated mouthwash to a predetermined amount of water, allowing the mouthwash to dissolve forming a solution, swishing the solution in the oral cavity, and expectorating the solution.

In one embodiment, the flexible porous dissolvable solid structure is in the form of a concentrated toothpaste. In a further embodiment, the concentrated toothpaste would be in the form a thin film which could be rehydrated by placing it in the mouth or by adding water to the it when on the brush. For example, a method of using a porous solid structured concentrated toothpaste would include putting the concentrated toothpaste in the oral cavity, allowing it to at least partially hydrate, and then brushing the teeth with a toothbrush. In a further embodiment, the toothpaste could be placed on at least a portion of the teeth in the oral cavity. In another embodiment, the method of use would include placing the concentrated toothpaste on a toothbrush, adding water to the concentrated toothpaste, and then brushing the teeth.

In another embodiment, the flexible porous dissolvable solid structure is in the form of a tooth whitening product. In a further embodiment, the porous solid structure tooth whitening product is placed directly on the teeth and a tooth whitening agent is released during hydration. In another embodiment, a tooth whitening composition is placed on top of the structure and the structure acts as a carrier. The concentrated tooth whitening product can be in any shape suitable for the oral cavity including but not limited to strips, dots, curved, arched, in the shape of a dental tray, etc. Additionally, the tooth whitening product may also contain a release liner and/or a backing layer. In one embodiment, the concentrated tooth whitening product is a liner which can be placed inside of a dental tray. In varying embodiments, the tooth whitening product is water soluble, partially water soluble, water insoluble, or a combination thereof. In one embodiment, a method for using a tooth whitening product includes placing the tooth whitening product on at least one tooth of a user. In a further embodiment, the method further comprises removing the tooth whitening product.

In yet another embodiment, the flexible porous dissolvable solid structure can be used for systemic active delivery through the oral cavity. In a further embodiment, the flexible porous dissolvable solid structure is a dissolvable wafer which releases active when hydrated in the oral cavity. In another embodiment, the flexible porous dissolvable solid structure is a partially water soluble wafer which will release product when manipulated in the mouth, for example, chewed.

In one embodiment, the flexible porous dissolvable solid structure can be used to make binders more easily dispersible. For example, traditional binders come in the form of powders. These powders are difficult to hydrate and thus must be hydrated prior to the addition of many other ingredients because poor or incomplete hydration can lead to a lumpy final product. By taking the binders and forming them into a water soluble structure, the binders are able to more quickly and completely hydrate and do not require any prehydration when making something containing the binder. In one embodiment, a binder is made into the form of a flexible porous dissolvable solid structure by dispersing the binder in a solvent, adding a polymer structurant, and drying the resulting mixture.

In an additional embodiment, the flexible porous dissolvable solid structure is used as an additive in an oral care composition. The use of pieces of flexible porous dissolvable solid structure as an additive in an oral care composition can be used, for example, as an abrasive to help clean teeth, as a carrier of oral care components that are reactive with other components in the oral care composition, as a flavor carrier, etc.

### Product Form

The flexible porous dissolvable solid structure can be produced in any of a variety of product forms, including porous dissolvable solid structures used alone or in combination with other consumer product components. The porous dissolvable solid structures can be used in a continuous or discontinuous manner when used within consumer product compositions. Regardless of the product form, the key to all of the product form embodiments contemplated within the scope of the method of the present invention is the selected and defined oral care article that comprises a combination of a solid water soluble polymeric structurant and an oral care component, all as defined herein.

The flexible porous dissolvable solid structure is preferably in the form of one or more flat sheets or pads of an adequate size to be able to be handled easily by the user. It may have a square, rectangle or disc shape or any other suitable shape. The pads can also be in the form of a continuous strip including delivered on a tape-like roll dispenser with individual portions dispensed via perforations and or a cutting mechanism. Alternatively, the structures are in the form of any other shaped object.

The flexible porous dissolvable solid structure may comprise one or more textured, dimpled or otherwise topographically patterned surfaces including letters, logos or figures. The textured substrate preferably results from the shape of the substrate, in that the outermost surface of the substrate contains portions that are raised with respect to other areas of the surface. The raised portions can result from the formed shape of the article, for example the article can be formed originally in a dimpled or waffle pattern. The raised portions can also be the result of creping processes, imprinted coatings, embossing patterns, laminating to other layers having raised portions, or the result of the physical form of the dissolvable fibrous solid substrate itself. The texturing can also be the result of laminating the substrate to a second substrate that is textured.

In a particular embodiment, the flexible porous dissolvable solid structure can be perforated with holes or channels penetrating into or through the fibrous solid. These perforations can be formed as part of the web making process via spikes extended from the surface of an adjacent belt, drum, roller or other surface. Alternatively, these perforations can be formed after the web making process via poking or sticking the porous solids with pins, needles or other sharp objects. Preferably, these perforations are great in number per surface area, but not so great in number so as to sacrifice the integrity or physical appearance of the porous solid. It has been found that such perforations increase the dissolution rate of the fibrous solids into water relative to un-perforated porous solids.

The flexible porous dissolvable solid structure can also be delivered via a water insoluble implement or device. For instance, they may be attached or glued by some mechanism to an applicator to facilitate application to hair, skin, fabric, hard surfaces, oral cavity, teeth and/or hard surfaces, i.e., a comb, rag, wand, or any other conceivable water-insoluble applicator. Additionally, the flexible porous dissolvable solid structure may be adsorbed to the surfaces a separate high surface area water-insoluble implement, i.e., a fibrous sponge, a puff, a flat sheet etc. For the latter, the flexible porous dissolvable solid structure of the present invention may be adsorbed as a thin film or layer.

### Method of Manufacture

In one embodiment, the flexible porous dissolvable solid structure can be prepared by the process comprising: (1) preparing a processing mixture with the desired components; (2) aerating the mixture by introducing a gas into the mixture; (3) forming the aerated wet mixture into a desired one or more shapes; and (4) drying the aerated wet mixture to a desired final moisture content. Optionally, a surface resident coating can be applied to the structure.

### Preparation of Processing Mixture

The processing mixture is generally prepared by dissolving the polymer structurant and other ingredients in the solvent. This may require heating depending upon the components. This can be accomplished by any suitable heated batch agitation system or via any suitable continuous system involving either single screw or twin screw extrusion or heat exchangers together with either high shear or static mixing. Any process can be envisioned such that the polymer is ultimately dissolved in the presence of the solvent, the surfactant(s), the plasticizer, and other ingredients including step-wise processing via pre-mix portions of any combination of ingredients. Once the ingredients are all dissolved and mixed, the processing mixture is cooled (if heating was required). In a preferred embodiment, the solvent is water and the polymeric structurant is water soluble as described herein.

The processing mixtures of the present invention comprise: from about 10% to about 70% solids, in one embodiment from about 20% to about 50% solids, and in another embodiment from about 25% to about 35% solids, by weight of the processing mixture before drying; and have a viscosity of from about 2,500 cps to about 150,000 cps, in one embodiment from about 5,000 cps to about 100,000 cps, in another embodiment from about 7,500 cps to about 50,000 cps, and in still another embodiment from about 10,000 cps to about 20,000 cps.

The % solids content is the summation of the weight percentages by weight of the total processing mixture of all of the solid, semi-solid and liquid components excluding water and any obviously volatile materials such as low boiling alcohols. The processing mixture viscosity values are measured using a TA Instruments AR500 Rheometer with 4.0 cm diameter parallel plate and 1,200 micron gap at a shear rate of 1.0 reciprocal second for a period of 30 seconds at 23° C.

### Optional Continued Heating of Pre-Mixture

Optionally, the pre-mixture is pre-heated immediately prior to the aeration process at above ambient temperature but below any temperatures that would cause degradation of the components. In one embodiment, the pre-mixture is kept at above about 40° C. and below about 99° C., preferably above about 50° C. and below about 95° C., more preferably about 60° C. and below about 90° C. In one embodiment, when the viscosity at ambient temperature of the pre-mix is from about 15,000 cps to about 150,000 cps, the optional continuous heating should be utilized before the aeration step. In an additional preferred embodiment, additional heat is applied during the aeration process to try and maintain an elevated temperature during the aeration. This can be accomplished via conductive heating from one or more surfaces, injection of steam or other processing means.

Without being limited by a theory, the act of pre-heating the pre-mixture before the aeration step provides a means for lowering the viscosity of pre-mixtures comprising higher percent solids content for improved introduction of bubbles into the mixture and formation of the desired porous solid structure. Achieving higher percent solids content is desirable so as to reduce the energy requirements for drying. The increase of percent solids, and therefore conversely the decrease in water level content, and increase in viscosity is believed to affect the bubble drainage from the pre-mixture during the drying step. The drainage and evaporation of water from the pre-mixture during drying is believed to be critical to the formation of the desired predominantly open-celled porous solid structure described herein.

Pre-heating of the pre-mixture enables the manufacture of the desired fast dissolving porous solid structure from more viscous processing mixtures with higher percent solids levels that would normally produce slow dissolving and predominantly closed celled porous structures. While not being bound to theory, the increased temperature is believed to influence controlled bubble drainage from the thin film bubble facings into the plateau borders of the three dimensional structure generating openings between the bubbles (formation of open-cells) simultaneous to the solidification of the resulting plateau border structure (driven by evaporation). The demonstrated ability to achieve such inter-connected open-celled solid structure architectures with good mechanical integrity and visual appearance of the article produced via the present invention and without collapse of the “unstable” structure during the drying process is surprising. The alternative predominantly closed celled porous solids that are typically produced without the processing innovations described herein have significantly poorer dissolution and do not meet the structural parameters encompassed by the porous solid structure described herein.

Moreover, the higher % solids and viscosity pre-mixtures resulted in solids with significantly less percent (%) shrinkage from the drying process while still resulting in porous solid structure with fast dissolution rates. On the one hand this is intuitive as the higher viscosities during the drying process should serve to mitigate the drainage and bubble rupture/collapse/coalescence that give rise to the shrinkage. However, on the other hand this is counterintuitive as such reduced drainage should mitigate the formation of the desired predominantly open-celled porous solid structure (with a minimum degree of cell interconnectivity) during the drying process.

### Aeration of Processing Mixture

The aeration of the processing mixture is accomplished by introducing a gas into the mixture, in one embodiment by mechanical mixing energy but also may be achieved via chemical means. The aeration may be accomplished by any suitable mechanical processing means, including but not limited to: (i) batch tank aeration via mechanical mixing including planetary mixers or other suitable mixing vessels, (ii) semi-continuous or continuous aerators utilized in the food industry (pressurized and non-pressurized), (iii) gas injection, (iv) gas evolution via pressure drop, or (v) spray-drying the processing mixture in order to form aerated beads or particles that can be compressed such as in a mould with heat in order to form the flexible porous dissolvable solid structure. The flexible porous dissolvable solid structure may also be prepared with chemical foaming agents by in-situ gas formation (via chemical reaction of one or more ingredients, including formation of CO₂ by an effervescent system).

In a particular embodiment, it has been discovered that the flexible porous dissolvable solid structure can be prepared within continuous pressurized aerators that are conventionally utilized within the foods industry in the production of marshmallows. Suitable continuous pressurized aerators include the Morton whisk (Morton Machine Co., Motherwell, Scotland), the Oakes continuous automatic mixer (E.T. Oakes Corporation, Hauppauge, N.Y.), the Fedco Continuous Mixer (The Peerless Group, Sidney, Ohio), and the Preswhip (Hosokawa Micron Group, Osaka, Japan).

The wet density range of the aerated pre-mixture ranges from about 0.10 g/cm³ to about 0.50 g/cm³, preferably from about 0.15 g/cm³ to about 0.45 g/cm³, more preferably from about 0.20 g/cm³ to about 0.40 g/cm³, and even more preferably from about 0.25 g/cm³ to about 0.35 g/cm³.

### Forming the Aerated Wet Processing Mixture

The forming of the aerated wet processing mixture may be accomplished by any suitable means to form the mixture in a desired shape or shapes including, but not limited to (i) depositing the aerated mixture into moulds of the desired shape and size comprising a non-interacting and non-stick surface including, for example, aluminium, Teflon, metal, HDPE, polycarbonate, neoprene, rubber, LDPE, glass and the like; (ii) depositing the aerated mixture into cavities imprinted in dry granular starch contained in a shallow tray, otherwise known as starch moulding forming technique; or (iii) depositing the aerated mixture onto a continuous belt or screen comprising any non-interacting or non-stick material Teflon, metal, HDPE, polycarbonate, neoprene, rubber, LDPE, glass and the like which may be later stamped, cut, embossed or stored on a roll.

### Drying the Formed Aerated Wet Processing Mixture

The drying of the formed aerated wet processing mixture may be accomplished by any suitable means including, but not limited to (i) drying room(s) including rooms with controlled temperature and pressure or atmospheric conditions; (ii) ovens including non-convection or convection ovens with controlled temperature and optionally humidity; (iii) Truck/Tray driers, (iv) multi-stage inline driers; (v) impingement ovens; (vi) rotary ovens/driers; (vii) inline roasters; (viii) rapid high heat transfer ovens and driers; (ix) dual plenum roasters, (x) conveyor driers, (xi) microwave driers, and combinations thereof. Additional examples of drying include freeze drying and extruding. In one embodiment, the drying is not done via freeze drying. In another embodiment, the drying is not done via extrusion.

In one embodiment, the drying environment is heated to a temperature between 40° C. and 150° C. In one embodiment, the drying temperature is between 75° C. and 145° C. In another embodiment, the drying temperature is between 100° C. and 140° C. In a further embodiment, the drying temperature is between 115° C. and 135° C.

It has been found that increasing the surrounding air temperature of the drying step to about 100° C. to about 150° C. decreases the drying time of the formed aerated wet pre-mixture in forming the article while maintaining the desired dissolution properties of the article. It has been found that increasing surrounding air temperature levels from ambient temperature (25° C.) to 40° C. produced a suitable article, but drying times to achieve a final moisture contents were several hours (typically requiring overnight drying). Increasing the surrounding air temperature of the drying step to 75° C. for a period of about 2 hours to the desired dry density provided an unsuitable article and producing a denser bottom region including the formation of continuous sticky film on the bottom surface (adjacent to the mold) of the formed solid with poorer dissolution. While not being bound to theory, it is believed that this denser bottom region and formed continuous film serve as a rate limiting barrier for water ingress thereby adversely affecting the dissolution performance of the overall porous solid.

Surprisingly, it was found that an increase in the surrounding air temperature above 75° C. for the drying step to about 100° C. to about 150° C. provides acceptable properties for the article within a 60 minute or less time frame while improving the desired dissolution properties. This is counter-intuitive given the poorer results observed upon increasing the temperature from 40° C. to 75° C. Moreover, this temperature range is above the boiling point of water and would thereby be expected to result in water vapour evaporation rates that likely exceed the rate of water vapour escape from the solid to the surrounding environment and resulting in the regional build-up of excessive internal solid pressure leading to increased expansion/thickness or “humped” cross sections of the resulting material. While not being bound to theory, it is believed that the initial aerated wet structure closed cells coalesce together during the critical stages of the drying process under these preferred temperature conditions, creating inter-connected open-celled channels extending to the surface of the solid, and thereby enabling the facile escape of the water vapour molecules without excessive pressure build-up and ensuing regional expansion of the resulting solid.

Increases in the drying temperature beyond 150° C. were generally found by the Applicants to lead to regional solid expansion as well as partial discoloration of the solid surface which is indicative of chemical decomposition at these elevated temperatures.

In another embodiment, it has been found that articles according to the present invention can be produced with a further improvement in the bottom region by Microwave drying. While not being bound to theory, it is believed that the internal heating afforded by Microwave heating technology helps to mitigate the drainage from the central region into the bottom region (adjacent to the mold surface) during the drying process and thereby creating a less dense bottom region and an overall structure with a more uniform density.

Importantly, microwave drying times of less than about 3 minutes result in undesired regional solid expansion of the article. While not being bound to theory, this is believed to be due to water vapour evaporation rates that exceed the rate of water vapour escape from the solid as described herein above. To achieve drying times beyond 3 minutes, Microwave drying is preferably achieved via a low energy density applicator such as are available via Industrial Microwave Systems L.L.C (Morrisville, N.C. http://www.industrialmicrowave.com/). In particular, a low energy two wide wave applicators in series microwave applicator system is preferred with two or more low energy applicator regions (about 5 kW). Ideally, the air environment within the low energy microwave applicator system is at an elevated temperature (typically from about 35° C. to about 90° C. and preferably from about 40° C. to about 70° C. and with good circulation so as to facilitate the removal of the resulting humidity.

In one embodiment, the drying time to the desired dry density is from about 3 minutes to about 90 minutes, in another embodiment from about 5 minutes to about 60 minutes, in another embodiment from about 7 minutes to about 45 minutes. The drying step results in the article. Drying times of less than about 3 minutes result in undesired regional solid cross-sectional expansion of the resulting article, whereas drying times beyond these values and up to 2 to 3 hours result in excessive densification of the bottom surface of the article leading to poorer dissolution. Drying times between 3 hours and 20 hours (overnight) lead to acceptable articles, but suffer from poorer economics of production.

The drying times that can be achieved via convective drying are between about 10 minutes to about 90 minutes, in another embodiment from about 20 minutes to about 60 minutes, and in another embodiment from about 30 minutes to about 45 minutes.

The drying times that can be achieved via Microwave drying are between about 3 minutes and about 25 minutes, in another embodiment between about 5 minutes and about 20 minutes, and in another embodiment between about 7 minutes and about 15 minutes.

### The Optional Preparing the Surface Resident Coating Comprising an Oral Care Component

The preparation of the surface resident coating comprising one or more oral care components may include any suitable mechanical, chemical, or otherwise means to produce a particulate composition comprising the oral care component(s) including any optional materials as described herein, or a coating from a fluid.

Optionally, the surface resident coating may comprise a water releasable matrix complex comprising oral care component(s). In one embodiment, the water releasable matrix complexes comprising oral care component(s) are prepared by spray drying wherein the oral care component(s) is dispersed or emulsified within an aqueous composition comprising the dissolved matrix material under high shear (with optional emulsifying agents) and spray dried into a fine powder. The optional emulsifying agents can include gum arabic, specially modified starches, or other tensides as taught in the spray drying art (See Flavor Encapsulation, edited by Sara J. Risch and Gary A. Reineccius, pages 9, 45-54 (1988), which is incorporated herein by reference). Other known methods of manufacturing the water releasable matrix complexes comprising oral care component(s) may include but are not limited to, fluid bed agglomeration, extrusion, cooling/crystallisation methods and the use of phase transfer catalysts to promote interfacial polymerisation. Alternatively, the oral care component(s) can be adsorbed or absorbed into or combined with a water releasable matrix material that has been previously produced via a variety of mechanical mixing means (spray drying, paddle mixers, grinding, milling etc.). In one embodiment, the water releasable matrix material in either pellet or granular or other solid-based form (and comprising any minor impurities as supplied by the supplier including residual solvents and plasticizers) may be ground or milled into a fine powder in the presence of the oral care component(s) via a variety of mechanical means, for instance in a grinder or hammer mill.

Where the flexible porous dissolvable solid structure has a surface resident particulate coating, the particle size is known to have a direct effect on the potential reactive surface area of the oral care components and thereby has a substantial effect on how fast the oral care component delivers the intended beneficial effect upon dilution with water. In this sense, the particulate surface resident coatings with smaller particle sizes tend to give a faster and shorter lived effect, whereas the surface resident coatings with larger particle sizes tend to give a slower and longer lived effect. In one embodiment the surface resident coatings of the present invention may have a particle size from about 1 μm to about 200 μm, in another embodiment from about 2 μm to about 100 μm, and in yet another embodiment from about 3 μm to about 50 μm.

In some embodiments, it is helpful to include inert fillers within the surface resident coatings as processing aides, for instance aluminum starch octenylsuccinate under the trade name DRY-FLO® PC and available from Akzo Nobel, at a level sufficient to improve the flow properties of the powder and to mitigate inter-particle sticking or agglomeration during powder production or handling. Other optional excipients or cosmetic actives, as described herein, can be incorporated during or after the powder preparation process, e.g., grinding, milling, blending, spray drying, etc. The resulting powder may also be blended with other inert powders, either of inert materials or other powder-active complexes, and including water absorbing powders as described herein.

In one embodiment, the oral care components may be surface coated with non-hygroscopic solvents, anhydrous oils, and/or waxes as defined herein. This may include the steps of: (i) coating the water sensitive powder with the non-hydroscopic solvents, anhydrous oils, and/or waxes; (ii) reduction of the particle size of the oral care component particulates, prior to, during, or after a coating is applied, by known mechanical means to a predetermined size or selected distribution of sizes; and (iii) blending the resulting coated particulates with other optional ingredients in particulate form. Alternatively, the coating of the non-hydroscopic solvents, anhydrous oils and/or waxes may be simultaneously applied to the other optional ingredients, in addition to the oral care components, of the surface resident coating composition and with subsequent particle size reduction as per the procedure described above.

Where the surface resident coatings are applied to the substrate as a fluid (such as by as a spray, a gel, or a cream coating), the fluid can be prepared prior to application onto the substrate or the fluid ingredients can be separately applied onto the substrate such as by two or more spray feed steams spraying separate components of the fluid onto the substrate.

### The Optional Combining of the Surface Resident Coating Comprising the Oral Care Components with the Flexible Porous Dissolvable Solid Structure

Any suitable application method can be used to apply the surface resident coating comprising oral care component(s) to the flexible porous dissolvable solid structure such that it forms a part of the article. For instance, the dissolvable structure can have a tacky surface by drying the porous dissolvable solid substrate's surface to a specific water content before application of powder to facilitate the adherence of the surface resident coating comprising the oral care components to the porous solid. In one embodiment, the dissolvable fibrous solid substrate is dried to a moisture content of from about 0.1% to about 25%, in one embodiment from about 3% to about 25%, in another embodiment from about 5% to about 20% and in yet another embodiment from about 7% to about 15%. Alternatively, a previously dried structure's surface can be made to reversibly absorb a desired level of atmospheric moisture prior to application of the powder within a controlled humidity environment for a specific period of time until equilibrium is achieved. In one embodiment, the humidity environment is controlled from about 20% to about 85% relative humidity; in another embodiment, from about 30% to about 75% relative humidity; and in yet another embodiment, from about about 40% to about 60% relative humidity.

In another embodiment, the dissolvable structure is placed in a bag, tray, belt, or drum containing or otherwise exposed to the powder and agitated, rolled, brushed, vibrated or shaken to apply and distribute the powder, either in a batch or continuous production manner. Other powder application methods may include powder sifters, electrostatic coating, tribo charging, fluidized beds, powder coating guns, corona guns, tumblers, electrostatic fluidized beds, electrostatic magnetic brushes, and/or powder spray booths. The surface resident coating comprising the oral care component(s) can be applied over portions or entire regions of the dissolvable fibrous solid substrate's exterior surface, and can be applied in a manner to adorn, decorate, form a logo, design, etc.

Where the coating is applied to the substrate as a fluid, it is preferable that if water is present in the fluid that the water is not sufficient to cause the substrate to undesirable dissolve. In preferred embodiments, the oral care component(s) to be applied as an adsorbed thin coating is an anhydrous or substantially anhydrous oil. Other non-water solvents, such as organic solvents which do not cause the substrate to dissolve may also be used. Any suitable application method can be used to apply the oral care component(s) in liquid form to the article such that it forms a surface-resident coating that is adsorbed to at least a portion of the solid/air interface of the article as a thin film. For instance, it can be sprayed, spread, dropped, printed, sandwiched between different articles or different portions of the same article, layered, injected, rolled on, or dipped. The oral care component(s) can be applied over portions or entire regions of the article's exterior surface, and can be applied in a manner to adorn, decorate, form a logo, design, etc.

Optional ingredients may be imparted during any of the above described four processing steps or even after the drying process.

### Examples

### Concentrated Toothpaste

### Example 1

| Component | Wt % |
|---|---|
| Distilled water | 65.0 |
| Glycerin | 4.0 |
| CELVOL ® 523 | 7.5 |
| Sodium Lauryl Sulfate (28% active) | 4.0 |
| Tween 80 | 7.0 |
| Flavor | 2.0 |
| Sodium Fluoride | 0.64 |
| Sodium Acid Pyrophosphate | 8.86 |
| Saccharin Sodium | 1.0 |
| Total | 100.0 |

¹ CELVOL ® 523 available from Celanese Corporation (Dallas, Texas)

### Concentrated Toothpaste with Abrasive

### Example 2

| Component | Wt % |
|---|---|
| Distilled water | 65.0 |
| Glycerin | 4.0 |
| CELVOL ® 523 | 7.5 |
| Sodium Lauryl Sulfate (28% active) | 4.0 |
| Tween 80 | 7.0 |
| Flavor | 2.0 |
| Sodium Fluoride | 0.64 |
| Sodium Acid Pyrophosphate | 3.86 |
| Silica | 5.0 |
| Saccharin Sodium | 1.0 |
| Total | 100.0 |

### Concentrated Mouthwash

### Example 3

| Component | Wt % |
|---|---|
| Distilled water | 77.38 |
| Glycerin | 4.0 |
| CELVOL ® 523 | 7.5 |
| Sodium Lauryl Sulfate (28% active) | 1.0 |
| Tween 80 | 7.0 |
| Flavor | 2.0 |
| Cetylpyridinium chloride | 0.12 |
| Saccharin Sodium | 1.0 |
| Total | 100.0 |

Examples 1-3 can be made with the following procedure. Into an appropriately sized and cleaned vessel, the distilled water and glycerin are added with stirring at 100-300 rpm. The CELVOL® 523 is weighed into a suitable container and slowly added to the main mixture in small increments using a spatula while continuing to stir while avoiding the formation of visible lumps. The mixing speed is adjusted to minimize foam formation. The mixture is slowly heated to 75° C. after which the sodium lauryl sulfate and Tween 80 are added. The mixture is allowed to again reach 75° C. The mixture is then heated to 85° C. while continuing to stir and then allowed to cool to room temperature. The remaining ingredients are added with mixing once the mixture is at room temperature. The viscosity of the mixture is approximately 10,000 to 15,000 cps at 1 s⁻¹.

250 grams of the above mixture is transferred into a 5 quart stainless steel bowl of a KITCHENAID® Mixer Model KSSS (available from Hobart Corporation, Troy, Ohio) and fitted with a flat beater attachment. The mixture is vigorously aerated at high speed for 30 seconds. A portion of the resulting aerated mixture is then spread with a spatula into 12 circular Teflon molds comprising of different sizes and shapes and placed into a 75° C. convection oven for 30 minutes and then placed into a 40° C. convection oven for drying overnight. The following day, the resulting porous solids are removed from the molds with the aid of a thin spatula and tweezers. The estimated surfactant level in the final product is about 23 wt % and the estimated polymer level is about 21 wt %. The final product may be used as a concentrated toothpaste.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care article in the form of a flexible porous dissolvable solid structure, comprising:
a. from about 1% to about 70%, by weight of the structure, of a surfactant;
b. from about 10% to about 70%, by weight of the structure, of a water soluble polymer;
c. an oral care component; and
d. from about 0% to about 25%, by weight of the structure, of a plasticizer;
wherein said article has a density of from about 0.03 g/cm³ to about 0.50 g/cm³.

2. An article according to claim 1 having a percent open-celled content of from about 80% to about 100%.

3. An article according to claim 1 wherein the structure has a wall thickness of from about 0.02 mm to about 0.15 mm.

4. An article according to claim 1 wherein the structure has a specific surface area of from about 0.01 m²/g to about 0.25 m²/g.

5. An article according to claim 1 wherein the structure has a basis weight of from about 125 grams/m² to about 1,500 grams/m².

6. An article according to claim 1 wherein the structure has a dry density of from about 0.05 g/cm³ to about 0.2 g/cm³,

7. An article according to claim 1 wherein the structure comprises multiple layers having different dissolution rates.

8. An article according to claim 1 wherein the article comprises from about 1% to about 25%, by weight of the structure, of the plasticizer.

9. An article according to claim 8 wherein the plasticizer is selected from glycerin, propylene glycol, or combinations thereof.

10. An article according to claim 1 wherein the surfactant is selected from anionic surfactants, nonionic surfactants, cationic surfactants, zwitterionic surfactants, amphoteric surfactants, and combinations thereof.

11. An article according to claim 10 wherein the surfactant is an anionic surfactant.

12. An article according to claim 1 wherein the water soluble polymer has a solubility in water of from about 0.1 gram/liter to about 500 grams/liter.

13. An article according to claim 1 wherein the water soluble polymer has a weighted average molecular weight of from about 60,000 to about 300,000.

14. An article according to claim 1 wherein the water soluble polymer is selected from polyvinyl alcohols, polyvinylpyrrolidones, polyalkylene oxides, starch, starch derivatives, pullulan, gelatin, hyroxypropylmethycelluloses, methycelluloses, carboxymethycelluloses, and combinations thereof.

15. An article according to claim 1 wherein the water soluble polymer is selected from polyvinyl alcohol, hydroxypropylmethylecllulose, or combinations thereof.

16. An article according to claim 1 wherein the article comprises from about 0.5% to about 80%, by weight of the structure, of the oral care component.

17. An article according to claim 1 wherein the oral care component is selected from flavors, colorants, sensates, sweeteners, metal salts, abrasives, salivation agents, refractive particles, anticaries agents, antimicrobial agents, anti-inflammatories, antierosion agents, antisensitivity agents, antitartar agents, whitening agents, hydrating agents, bad breath reduction agents, bleaching agents, and combinations thereof.

18. The article of claim 1 comprising a surface resident coating comprising from about 1% to about 70%, by weight of the article, of one or more oral care components and wherein the ratio of the porous dissolvable solid substrate to the surface resident coating comprising said at least one oral care component is from about 110:1 to about 0.1:1.

19. The article of claim 18, wherein the surface resident coating comprises from about 10% to about 100% of one or more water-releasable matrix complexes comprising one or more oral care components.

20. The article of claim 19, wherein the water-releasable matrix materials are selected from cyclodextrins, as well as high surface area particles that form complexes such as starches, polyethylenes, polyamides, polystyrenes, polyisoprenes, polycarbonates, polyesters, polyacrylates, vinyl polymers polyurethanes, amorphous silica, amorphous silica gel, precipitated silica, fumed silica, aluminosilicates, such as zeolites and alumina, silicates, carbonates, and mixtures thereof.

21. The article of claim 19, wherein the surface resident coating comprises from about 10% to about 100% of one or more microcapsules comprising active agents.

22. A process of making an oral care article wherein the article is in the form of a flexible porous dissolvable solid structure, said process comprising the steps of:
a. preparing a pre-mix comprising surfactant, dissolved polymer structurant, and optionally plasticizer, wherein said pre-mix has: from about 10% to 70% total solids and a viscosity of from about 2,500 cps to 150,000 cps;
b. aerating said pre-mix by introducing a gas into the pre-mix to form a wet aerated pre-mix;
c. forming the wet aerated pre-mix into a desired one or more shapes to form shaped wet pre-mix; and
d. drying the shaped wet pre-mix to a desired final moisture content of from about 0.5% to about 15% moisture, to form the oral care article.
